(19) United States
(12) Patent Application Publication
(10) Pub. No.: US 2006/0247420 A1
(43) Pub. Date: Nov. 2, 2006

(54) Immune cell recptor ligand and immune cell receptor

(76) Inventors: George Coukos, Philadephia, PA (US); Jose Conejo-Garcia, Philadelphia, PA (US)

Correspondence Address: NIXON & VANDERHYE, PC, 901 NORTH GLEBE ROAD, 11TH FLOOR, ARLINGTON, VA, 22203, US

(21) Appl. No.: 10/525,643

(22) PCT Filed: Sep. 4, 2003
(86) PCT No.: PCT/US03/27488

§ 371 Date: Mar. 6, 2006

Related U.S. Application Data
(60) Provisional application No. 60/408,397, filed on Sep. 4, 2002.
(60) Provisional application No. 60/478,371, filed on Jun. 13, 2003.

(51) Int. Cl. C07K 14/705; C07K 16/28; A61K 38/17; C07H 21/04; C12P 21/06

(52) U.S. Cl. 530/350; 435/69.1; 435/320.1; 435/325; 536/23.5; 530/388.22; 514/12

(57) ABSTRACT

The present invention relates, in general, to immune cell receptor ligands and immune cell receptors. More specifically, the invention relates to an NKG2D immunoreceptor ligand and to an immune cell receptor having the same C-type lectin structure as the NKG2D receptor, and to nucleic acid sequences encoding same.

This application claims priority from Provisional Application No. 60/408,397, filed Sep. 4, 2002, and Provisional Application No. 60/478,371, filed Jun. 13, 2003, the contents of these Provisional Applications being incorporated herein by reference.

### TECHNICAL FIELD

The present invention relates, in general, to immune cell receptor ligands and immune cell receptors. More specifically, the invention relates to an NKG2D immunoreceptor ligand and to an immune cell receptor having the same C-type lectin structure as the NKG2D receptor, and to nucleic acid sequences encoding same.

### BACKGROUND

Induction of tumor-specific cytotoxic lymphocytes (CTLs) by tumor antigens presented in the context of class-I MHC molecules and the activation of natural killer (NK) cells play a critical role in antitumor immune response (Yee and Greenberg, Nat. Rev. Cancer 2(6):409-419 (2002), Pardoll, Science 294(5542):534-536 (2001)). The balance between activating and inhibitory ligands expressed in tumors may critically affect the function of effector lymphocytes and the efficacy of antitumor immune response.

NKG2D is expressed on most CD8⁺ T-cells, γδ T-cells and NK cells and serves as one of the most potent activating receptors for effector lymphocytes in peripheral tissues (Bauer et al, Science 285(5428):727-729 (1999), Groh et al, Nat. Immunol. 2(3):255-260 (2001)). NKG2D polypeptides associate with the adaptor molecule DAP10 (and DAP12 in the mouse), providing a costimulatory signal to CD8⁺ lymphocytes, or a primary stimulatory signal to NK cells, respectively (Diefenbach et al, Nature 413(6852):165-171 (2001), Jamieson et al, Immunity 17(1):19-29 (2002), Lanier et al, Nature 391(6668):703-707 (1998)). NKG2D ligands mediate destruction of virus-infected cells and mark tumor cells for cell-mediated killing (Bauer et al, Science 285(5428):727-729 (1999), Cosman et al, Immunity 14(2):123-133 (2001)). For example, in the mouse, ectopic expression of the NKG2D ligands Rae1b or H60 in tumor cell lines has resulted in cell-mediated rejection of tumors (Diefenbach et al, Nature 413(6852):165-171 (2001), Cerwenka et al, Proc. Natl. Acad. Sci. USA 98(20):11521-11526 (2001)). Furthermore, skin-associated NKG2D⁺ γδ T-cells successfully mediate destruction of carcinoma cells in vivo, utilizing a mechanism dependent on NKG2D receptor engagement (Girardi et al, Science 294(5542):605-609 (2001)).

Several human and murine molecules related to class-I major histocompatibility complex (MHC) molecules have been identified as ligands for NKG2D. In humans, the ligands for NKG2D fall into either the MIC group or the UL16-binding protein (ULBP) group. The MIC group consists of MICA and MICB, which are closely related. Both are encoded within the human MHC locus and are expressed on a wide range of epithelial tumors. MICA and MICB are stress-inducible molecules which trigger NK cell activation and function as costimulatory ligands that can substitute for B7 ligands (Bauer et al, Science 285(5428):727-729 (1999)). The ULBP group consists of ULBP1, ULBP2 and ULBP3, which were identified based on their ability to bind to the human cytomegalovirus glycoprotein UL16 (Cosman et al, Immunity 14(2):123-133 (2001)).

The present invention results, at least in part, from the characterization of a tumor-associated MHC-I related ligand for the NKG2D receptor, designated herein as Lymphocyte Effector cell Toxicity Activating Ligand (Letal). Letal acts as a costimulator in CD8⁺ CTLs, inducing their expansion and activation. Letal also induces cytotoxicity in NK cells. Another embodiment of the invention results from the identification of an immune cell receptor having the same C-type lectin structure as the NKG2D receptor.

### SUMMARY OF THE INVENTION

The present invention relates generally to immune cell receptor ligands and immune cell receptors. More specifically, the invention relates to an NKG2D receptor ligand (designated “Letal”). The invention further relates to an immune cell receptor that has the same C-type lectin structure as the NKG2D receptor. The invention additionally relates to nucleic acid sequences encoding the ligand and the receptor.

Objects and advantages of the present invention will be clear from the description that follows.

### BRIEF DESCRIPTION OF THE DRAWINGS

FIGS. 1A-1F. Letal is a new NKG2D ligand exhibiting a cytoplasmic domain. FIG. 1A, The genomic sequence of Letal reveals the presence of 4 exons separated by 3 short introns, the sequences of the 4 exons being set forth in FIG. 1E (SEQ ID NOs:1-4) and a depiction of the translation of the cDNA into the amino acid sequence being set forth in FIG. 1F. FIG. 1B, Alignment of Letal (SEQ ID NO:8) and ULBPs (SEQ ID NOs:5-7). The transmembrane segment, spanning amino acids from 226 to 248, is marked with asterisks. FIG. 1C, Phylogeny of all the murine and human NKG2D ligands so far characterized, generated with the topological algorithm of ClustalW. FIG. 1D, Letal is a ligand for NKG2D. All the results shown are representative of at least 3 experiments. Left: Letal-transduced K562 cells exhibit a much stronger staining by a polyclonal anti-Letal Ab than mock-transductants. Incubation with GPI-specific Phospholipase C results in slight increase in binding, suggesting a positive effect in exposing epitopes, and confirming the transmembrane structure of Letal. Shaded: Letal⁺ transductants; open thick: Mock-transductants; dotted: Treatment with Phospholipase C. Center, right: Control or Letal-transduced K562 and SKOV3 cells were incubated with a NKG2D-Fc protein, and stained with anti-human Ig mAb. Shaded: Letal⁺ transductants; open thick: Mock-transductants. When Letal⁺-K562 cells are treated with GPI-specific Phospholipase C, sNKG2D binding decreases, but it is still stronger than that of mock transductants, corresponding to the abundant expression of GPI-anchored NKG2D ligands in K562 cell line (dotted). No significant decrease is observed with SKOV3.

FIGS. 2A and 2B. Expression of Letal and NKG2D in different normal tissues and tumor cell lines. FIG. 2A, Letal is expressed by a variety of normal tissues, as revealed by TaqMan analysis. FIG. 2B, total RNA from several ovarian and colon cancer cell lines was subjected to RT-PCR using specific primers for Letal. The specificity of the products was confirmed by sequence analysis.

FIGS. 3A-3C. Regulation of Letal and NKG2D in tumor cells and lymphocytes. FIG. 3A, analysis of the effect of viral infection and inflammatory mediators on Letal expression in ovarian carcinoma cells A2008. FIG. 3B, Retinoic acid treatment induces a progressive decrease in Letal mRNA expression. Results are representative of 3 experiments. FIG. 3C, up-regulation of NKG2D by fresh peripheral blood lymphocytes upon Letal engagement. Shaded: Unstimulated CD8⁺ cells.

FIGS. 4A-4C. Effects of Letal on CD8⁺ lymphocytes. Data are representative of at least 3 experiments performed. FIG. 4A, Triggering of peripheral CD8⁺ T-cells with K32-bound anti-CD3 results in stronger proliferation in the presence of Letal or anti-CD28, as measured by [³H] thymidine incorporation. FIGS. 4B, 4C, Combined triggering with anti-CD3 and Letal or CD28 induces significant differences in IL-2 and IFN-γ secretion by CD8⁺ T-cells compared to anti-CD3 signaling alone.

FIGS. 5A and 5B. Expression of Letal induces the killing of cancer cells by CD8⁺ and NK cells. FIG. 5A, Letal induces anti-tumor lymphocyte cytotoxicity. Letal alone shows a modest effect in redirecting cytotoxicity against Letal⁺ K32 erythroleukemia cells by peripheral CD8⁺ T-cells activated for 3 days with anti-CD3/Letal. Addition of anti-CD3 mAb (0.5 μg/ml) increases specific lysis (x=effector:target ratio). FIG. 5B, Ectopic expression of Letal increases NK cell-mediated cytotoxicity of the Letal⁻, p53⁻, chemoresistant ovarian cancer cell line SKOV3.

FIGS. 6A-6F. Immunohistochemical staining of advanced human ovarian carcinomas. Nuclei were counterstained with hematoxylin. These images are representative of different ovarian carcinomas, showing: FIG. 6A, high frequency of CD45⁺ leukocytes stained with anti-CD45 mAb (magnification: X20); FIG. 6B, high proportion of NKG2D+lymphocytes in most specimens analyzed (magnification: X10); FIG. 6C, a high frequency of tumor-infiltrating CD8⁺ cells is noted; in average, these cells represented 15% of total leukocytes (magnification: X20) FIG. 6D, CD57⁺ NK cells are only occasionally present (less than 1% of total CD45⁺ cells), indicating a predominant role of T-cell-mediated responses against advanced carcinomas; FIGS. 6E, 6F, expression of Letal in tumor islets of stage III ovarian carcinomas. Letal stainin is also noted on tumor-infiltrating leukocytes.

FIGS. 7A-7D. Letal and GLPD1 expression in normal and neoplastic ovarian tissues. FIG. 7A, Quantification of Letal mRNA levels by TaqMan in human normal ovaries and benign tumors (n=6); borderline tumors (n=4); stage I (n=9), and stage III ovarian carcinomas (n=29). FIG. 7B, Letal mRNA expression analyzed by TaqMan PCR in tumor islets isolated by laser capture microdissection. 12 specimens were evaluated with CD3⁺ cells infiltrating tumor islets and 7 with no T-cells in tumor islets. FIG. 7C, Kaplan-Meier curves for the duration of overall survival, according to the presence or absence of Letal mRNA in 38 patients with stage III epithelial ovarian cancer. Letal expression was analyzed by Real-Time PCR. P values were derived with the use of log-rank statistic. FIG. 7D, Quantification of GLPD1 mRNA levels by TaqMan in the same specimens. Results are expressed as number of copies of the gene of interest per each 10⁶ GAPDH copies.

FIGS. 8A-8C. Letal induces a sustained expansion of tumor-inflitrating CD28⁻ effector lymphocytes. FIG. 8A, Most CD8⁺ lymphocytes in solid tumors and ascites do not express the costimulatory molecule CD28. Gate on CD8⁺ cells. FIG. 8B, Sustained expansion of sorted CD8⁺ CD28⁻ lymphocytes through CD3/Letal engagement. Left, tumor-inflitrating lymphocytes; right, lymphocytes sorted from tumor ascites. FIG. 8C, Combined triggering with anti-CD3 and Letal induced a dramatic increase in IFN-γ secretion by CD28⁻CD8⁺ 5 days after the third cycle of stimulation. Results are compared to a pool of supernatants from the same cells activated with anti-CD3 signaling alone.

FIGS. 9A-9C. Letal engagement protects lymphocytes from cisplatin-induced apoptosis. FIGS. 9A, 9B, Letal stimulation induces Glut-1 and increases glucose uptake by CD8⁺ lymphocytes. Letal⁻ K32 cells were used as a non-stimulatory control. Shaded: Lymphocytes stimulated with CD3-alone. Total cellular Glut-1 was measured by flow cytometry; glucose uptake was evaluated with [³H]-2-deoxyglucose. FIG. 9C, Letal engagement protects CD8⁺ lymphocytes from genotoxic drugs. Peripheral CD8⁺ T-cells were stimulated for 3 days with the indicated factors and then incubated with cisplatin. Results are expressed as percentage of apoptotic cells. All the results are representative of at least 3 experiments.

FIGS. 10A-10C. Letal engagement protects lymphocytes from Fas-dependent apoptosis. FIG. 10A, Selected ovarian carcinoma specimens exhibit intense FasL staining in cells by immunohistochemistry. FIG. 10B, Downregulation of Fas by peripheral blood lymphocytes upon CD3/Letal engagement. Lymphocytes were stimulated for 4 days with the indicated conditions, and Fas expression was analyzed by flow cytometry. Shaded: Unstimulated CD8⁺ cells. FIG. 10C, Letal stimulation induces resistance to FasL-dependent apoptotic death. CD8⁺ lymphocytes treated with the indicated factors for 4 days were exposed to anti-Fas Ab that delivers an apoptotic signal to Fas-sensitive cells. More than 25% of Letal-stimulated lymphocytes resist apoptosis after 18 h. A representative analysis of 3 experiments is shown. Results are expressed as percentage of non-apoptotic cells.

FIG. 11. cDNA sequence of immune cell receptor LCCR (SEQ ID NO:9).

FIG. 12. Predicted protein sequence of immune LCCR cell receptor (SEQ ID NO:10).

### DETAILED DESCRIPTION OF THE INVENTION

The present invention relates, in one embodiment, to an MHC-1 related ligand for the NKG2D receptor, and fragments and variants thereof, and to nucleic acid sequences encoding same. The ligand, which provides a costimulatory signal to CD8⁺ lymphocytes (inducing their proliferation and activation), is upregulated in certain tumors and induces cytotoxicity in NK cells.

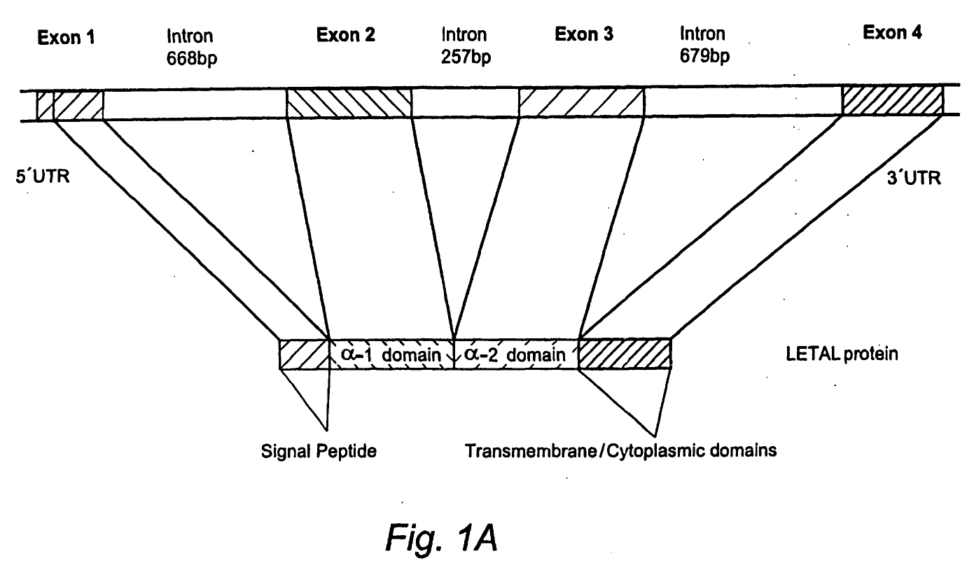

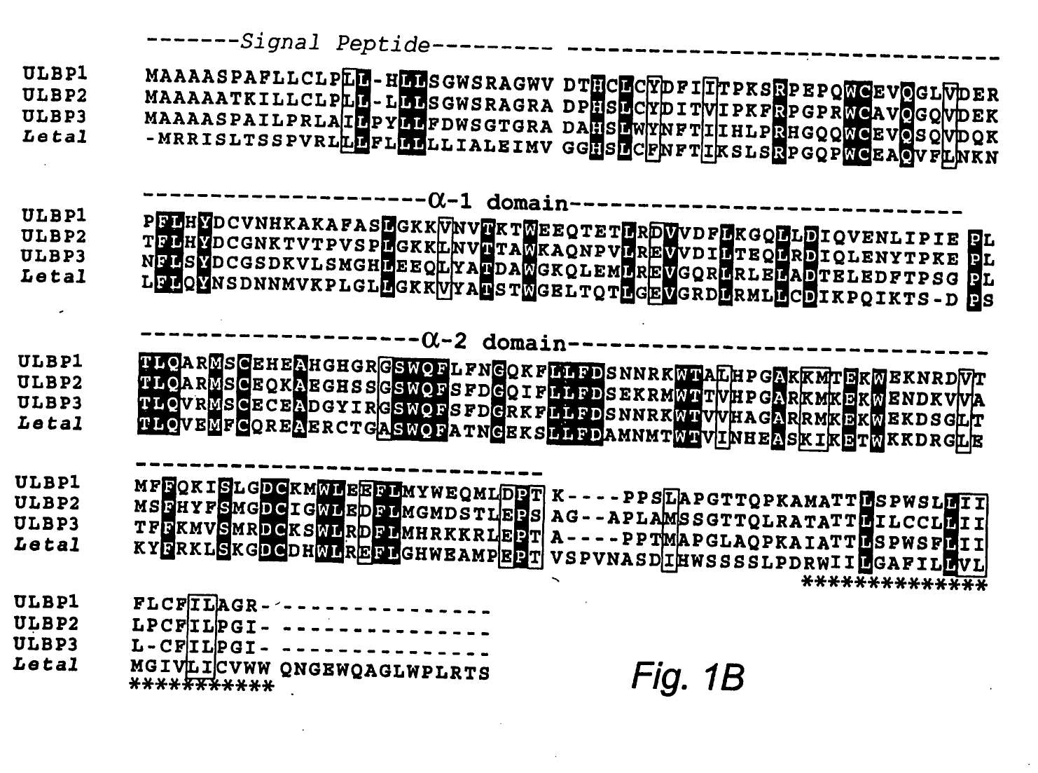

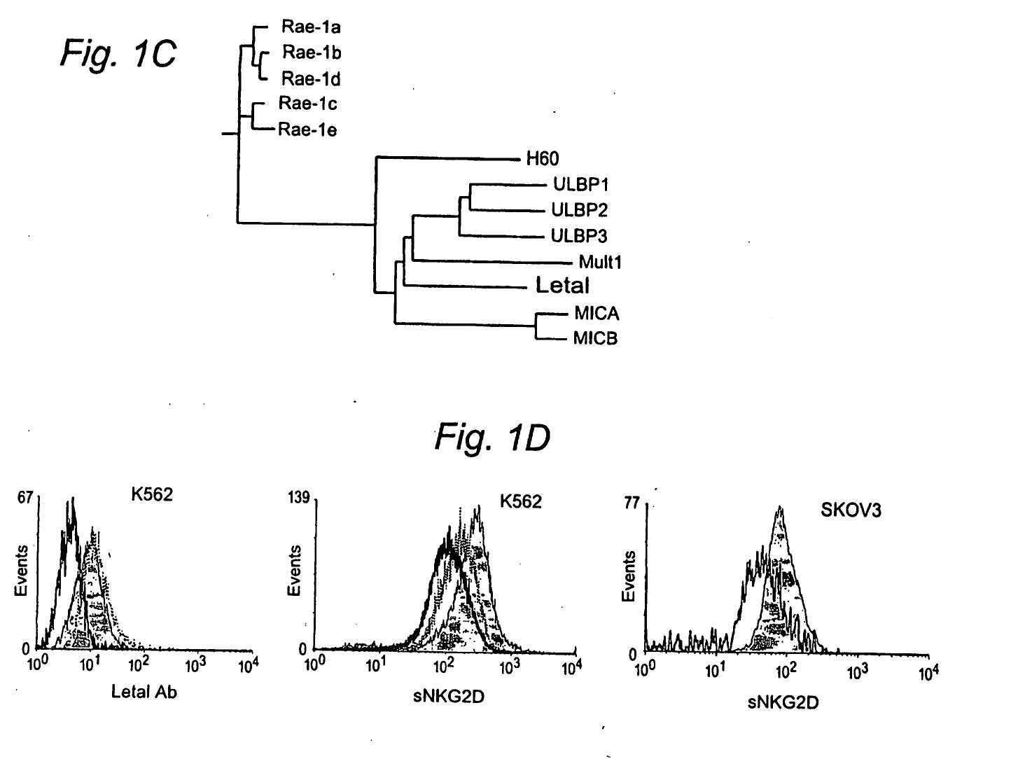

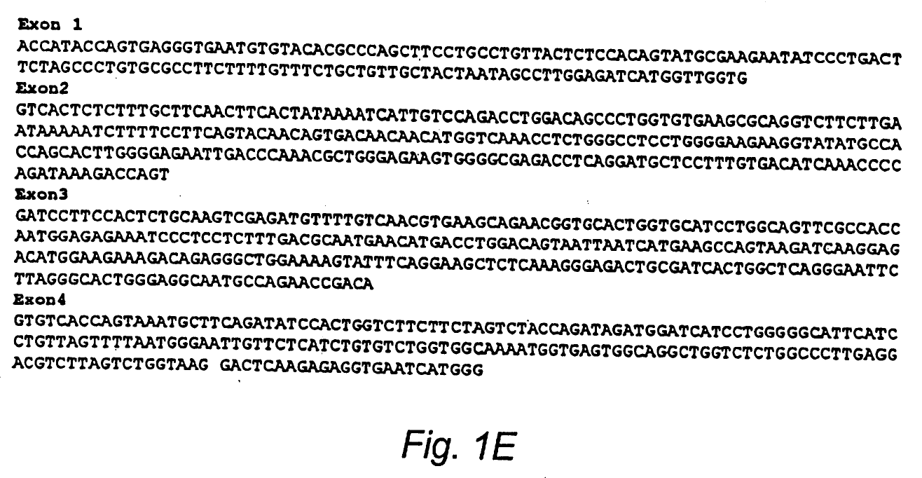

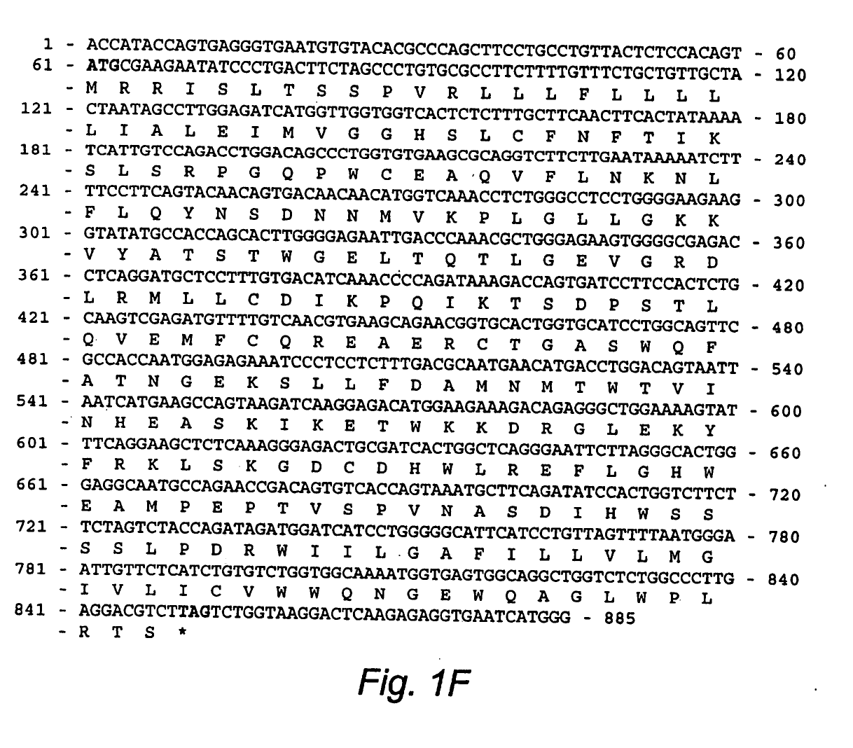

In a specific embodiment, the ligand is a polypeptide having the amino acid sequence set forth in SEQ ID NO:8 (see also FIGS. 1B and 1F), which polypeptide is referred to herein as “Letal”. This same polypeptide is referred to as “ULBP4” by Chalupny et al (Bioch. Biophys. Res. Commun. 305:129 (2003)). The invention includes this specific polypeptide and variants thereof, as well as fragments thereof. The invention also includes analogs/derivatives of such sequences.

Letal variants of the invention include polypeptides substantially identical to the sequence of SEQ ID NO:8. Letal variants of the invention do not include ULBP1, 2 or 3. The variants can include one or more deletions, insertions, or substitutions relative to the sequence of SEQ ID NO:8 (e.g., substitutions of one or more of the amino acids of SEQ ID NO:8 wherein the substitution is with a conserved or non-conserved amino acid (preferably, a conserved amino acid)). The variant can have an amino acid sequence that is, for example, at least 50%, at least 60% or at least 70% identical to the sequence of SEQ ID NO:8, at least 80% identical, least 90% identical, at least 95% identical, at least 98% identical, at least 99% identical, or at least 99.9% identical to the sequence of SEQ ID NO:8. The percent identity can be determined, for example, by comparing sequence information using BLAST 2 SEQUENCES. Variants in which differences in amino acid sequence relative to the sequence of SEQ ID NO:8 are attributable to genetic polymorphism (allelic variation among individuals producing the protein) are within the scope of the invention. Preferred variants include the residues at the positions bolded in FIG. 1B that are common to Letal and the depicted ULBP sequences.

Fragments of the invention include, but are not limited to, peptides/polypeptides comprising the signal peptide (e.g., about amino acid 1 to about amino acid 28 of the sequence of SEQ ID NO:8), the α-1 domain (e.g., about amino acid 29 to about amino acid 116 of the sequence of SEQ ID NO:8), the α-2 domain (e.g., about amino acid 117 to about amino acid 207 of the sequence of SEQ ID NO:8), the transmembrane domain (e.g., about amino acid 226 to about amino acid 248 of the sequence of SEQ ID NO:8), the cytoplasmic domain (e.g., about amino acids 249 to 263 of the sequence of SEQ ID NO:8) of the Letal polypeptide of SEQ ID NO:8, or variants thereof. The invention also includes fragments of the polypeptide of SEQ ID NO:8, preferably, fragments comprising at least 5 consecutive amino acids, more preferably, at least 10 or at least 20 consecutive amino acids of the sequence of SEQ ID NO:8, or variant thereof. It will be appreciated that fragments of the invention can be employed as immunogens, in generating antibodies (monoclonal and polyclonal) using standard techniques.

Variants and fragments of the invention include, but are not limited to, polypeptides that retain a biological activity of the Letal polypeptide, for example, the ability to bind NKG2D receptor. An example of such a polypeptide is a soluble fragment of the sequence of SEQ ID NO:8, or variant thereof. Such soluble polypeptides include, but are not limited to, polypeptides comprising about amino acid 29 to about amino acid 225 of the sequence of SEQ ID NO:8, or variant thereof. Polypeptides of the invention can be tested for the ability to bind the NKG2D receptor in any suitable assay, such as a conventional binding assay. To illustrate, the polypeptide can be labeled with a detectable reagent (e.g., a radionuclide, chromophore, enzyme that catalyzes a colorimetric or fluorometric reaction, etc.). The labeled polypeptide can be contacted with cells expressing NKG2D receptor. The cells can then be washed to remove unbound labeled polypeptide, and the presence of cell-bound label can be determined by a suitable technique, chosen according to the nature of the label.

In certain aspects, the sequences of GenBank accession numbers AY054974 and AF359243 may not be within the scope of the invention.

The invention also includes derivatives/analogs of the Letal polypeptide of SEQ ID NO:8 and variants and fragments thereof. For example, the invention includes polypeptides in which one or more of the amino acid residues is fused with another compound, such as a compound to increase the half-life of the polypeptide (for example, polyethylene glycol). The invention also includes glycosylated polypeptides, cyclic polypeptides and polypeptides bearing a detectable label and/or bound to a solid support. The polypeptides can also include tumor-binding moieties, that is the invention includes chimeric molecules (e.g., bispecific) that include a Letal domain and an antibody variable domain directed against a tumor specific epitope (e.g., folate binding protein or CA125 (ovarian tumors)). The polypeptides of the invention can also be present as a fusion protein, for example, to facilitate detection or isolation.

The invention includes isolated and purified, or homogeneous, polypeptides, both recombinant and non-recombinant. The polypeptides can be synthesized chemically using art recognized techniques. The polypeptides can be used as described below or can be used in the production of antibodies (polyclonal or monoclonal) using standard techniques. The invention includes such antibodies, and binding portions thereof, as well as their use, for example, in detecting the presence of a polypeptide of the invention in a sample (in which case, the antibody can bear a detectable label).

The invention further relates to nucleic acid sequences encoding the sequence of SEQ ID NO:8, or variants, and fragments thereof, or the complements of such encoding sequences. One specific nucleic acid sequence encoding the amino acid sequence of SEQ ID NO:8 is that set forth in SEQ ID Nos:1-4 (see also FIGS. 1E and 1F). DNAs of the invention can be single or double stranded.

As indicated above, the invention includes encoding sequences (DNA and RNA) and sequences complementary thereto. Such complementary sequences include those that hybridize to a nucleic acid sequence encoding the sequence of SEQ ID NO:8, or variant thereof, or fragment thereof, under conditions of moderate or high stringency. As used herein, conditions of moderate stringency can be readily determined by those having ordinary skill in the art based on, for example, the length of the DNA. Conditions are set forth by Sambrook et al, Molecular Cloning: A Laboratory Manual, 2 ed. Vol. 1, pp. 1.101-104, Cold Spring Harbor Laboratory Press, (1989), and include use of a prewashing solution for the nitrocellulose filters 5×SSC, 0.5% SDS, 1.0 mM EDTA (pH 8.0), hybridization conditions of about 50% formamide, 6×SSC at about 42° C., and washing conditions of about 60° C., 0.5×SSC, 0.1% SDS. Conditions of high stringency can also be readily determined by the skilled artisan based on, for example, the length of the DNA. Generally, such conditions are defined as hybridization conditions as above, and with washing at approximately 68° C., 0.2×SSC, 0.1% SDS. The artisan will recognize that the temperature and wash solution salt concentration can be adjusted as necessary according to factors such as the length of the probe.

The invention also includes nucleic acids comprising sequences that are at least 50%, 60%, 70%, 80%, 90%, 95%, 98%, 99%, or at least 99.9% identical to the sequence of SEQ ID Nos:1-4. The percent identity can be determined by visual inspection and mathematical calculation. Alternatively, the percent identity of two nucleic acids can be determined by comparing sequence information using BLAST 2 SEQUENCES.

The nucleic acids can bear a detactable label and/or can be bound to a solid support.

The present invention also relates to a recombinant molecule comprising a nucleic acid as described above and to a host cell transformed therewith. Using standard methodologies, well known in the art, a recombinant molecule comprising a vector and a nucleic acid encoding a polypeptide of the invention can be constructed. Vectors suitable for use in the present invention include plasmid and viral vectors (e.g., advenoviral, adeno-associated or retroviral vectors). Vectors into which a nucleic acid can be cloned include any vectors compatible with transformation into a selected host cell. The nucleic acids of the invention can be present in the vector operably linked to regulatory elements, for example, a promoter. Suitable promoters include, but are not limited to the telomerase promoter, tumor specific promoters (e.g., ovarian cancer—MISIIR promoter, colorectal cancer—CEA promoter, prostate cancer—PSA promoter).

As indicated above, the recombinant molecule of the invention can be constructed so as to be suitable for transforming a host cell. Suitable host cells include prokaryotic cells (e.g., bacterial cells) and lower (e.g., yeast) and higher eucaryotic cells (e.g., mammalian cells, such as human cells). The recombinant molecule of the invention can be introduced into appropriate host cells by one skilled in the art using a variety of known methods.

The present invention further relates to a method of producing a polypeptide of the invention. In one aspect, the method comprises culturing the above-described transformed host cells under conditions such that the encoding sequence is expressed and the protein thereby produced.

The functional potency of Letal to stimulate effector immune cells and increase NKG2D expression makes possible new therapeutic strategies. Letal, and functional variants and fragments thereof, can be used, for example, to enhance proliferation of immunoeffector cells (e.g., NK cells and NKT cells) and/or CTL activity both in vitro and in vivo and thereby modulate an immune response, for example, against tumors and infectious agents (e.g., viruses and bacteria). In a preferred embodiment, Letal, or functional variants or fragments thereof, can be used to expand in vitro reactive T-cells or other effector cells, such as TALL cells, for use in adoptive immunotherapy for patients with cancer and infectious (e.g., viral) diseases. In one approach to such therapies, artificial antigen presenting cells expressing Letal and coated with anti-CD3 antibodies or specific tumor antigens can be used (see Maus et al, Nature Biotechnology 20:143 (2002)) in order to overcome the difficulty in obtaining sufficient numbers of CTLs. In the case of cancer immunotherapy, tumor cells and tumor infiltrating lymphocytes can be isolated from a patient. Following transduction of the tumor cells with a Letal (or Letal variant or fragment) encoding sequence, the transduced cells can be incubated with the isolated T cells to expand the population of T cells recognizing tumor antigen. The resulting expanded population of tumor specific T cells can then be administered (e.g., i.v.) to the patient to promote NK cytotoxicity and provide costimulatory signals to CTL through NKG2D interactions. (As regards details of these types of therapeutic approaches, see generally Liebowitz et al, Curr Opin Oncol. 10(6):533-41 (1998) and U.S. Appln. No. 20020187151)).

It will be appreciated from a reading of this disclosure that, in the treatment of autoimmune diseases (including rheumatoid arthritis), and in controlling rejection in patients undergoing tissue or organ transplantation, inhibition of expression or function of Letal can be useful. In this regard, siRNA technology, for example, can be used to block Letal expression and blocking agents (e.g., agents that block binding of Letal to the receptor) can be used to inhibit Letal function (e.g., blocking antibodies).

In addition to the foregoing, it will be apparent to one skilled in the art that Letal can serve as a marker for at least certain cancers. By assaying for the presence of Letal (e.g., using anti-Letal antibodies), for example, in a tissue sample, the presence of at least certain tumors can be detected. Letal detection can also be used as a means to monitor residual or recurrent disease after treatment. Methods of using markers such as Letal for cancer detection are well known in the art.

Similarly, it will be appreciated that the nucleic acid sequences of the invention can be used as probes and primers in detecting the presence Letal genes or gene transcripts. Such detection can be useful, for example, in cancer diagnosis.

In a further embodiment, the present invention relates to a previously unidentified molecule having the same C-type lectin structure as the NKG2D receptor. The encoding sequence for this molecule, designated herein as LCCR, maps at chromosome 12, in the same cluster as the NKG2D receptor. As the NKG2D receptor, which is present on most CD8+ T-cells, γδ T-cells and NK cells, induces cytotoxicity by interacting with ligands on the surface of tumor cells and cells infected by virus, it is expected that LCCR also interacts with tumor cell ligands and/or ligands on the surface of virus infected cells and induces cytotoxicity against them.

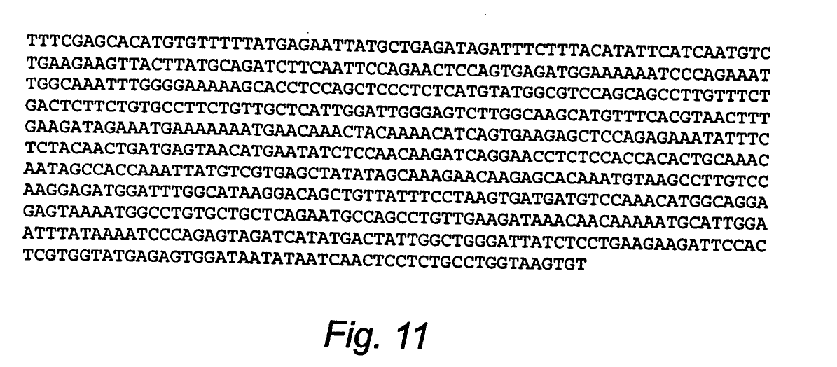

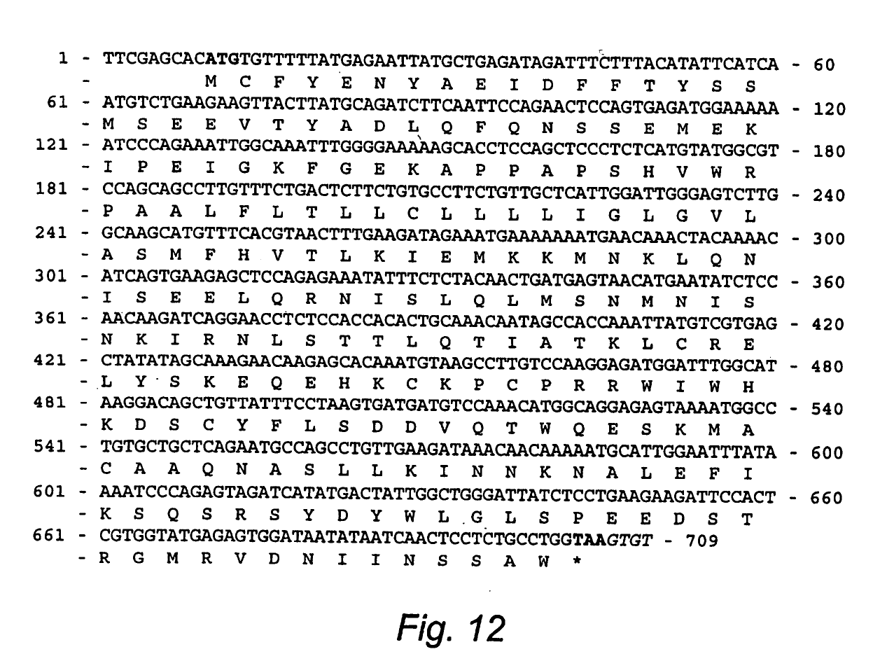

A cDNA sequence encoding LCCR is as shown in FIG. 11 (SEQ ID NO:9) and the predicted protein is shown in FIG. 12 (SEQ ID NO:10).

The invention includes the specific polypeptide shown in SEQ ID NO:10 and variants and fragments thereof, as well as analogs and derivatives of such sequences.

LCCR variants include polypeptides substantially identical to the sequence of SEQ ID NO:10. The variants can include one or more deletions, insertions, or substitutions relative to the sequence of SEQ ID NO:10 (e.g., substitutions of one or more of the amino acids of SEQ ID NO:10 wherein the substitution is with a conserved or non-conserved amino acid). The variant can have an amino acid sequence that is, for example, at least, 50%, 60% or 70% identical to the sequence of SEQ ID NO:10, at least 80% identical, least 90% identical, at least 95% identical, at least 98% identical, at least 99% identical, or at least 99.9% identical to the sequence of SEQ ID NO:10. The percent identity can be determined, for example, by comparing sequence information using BLAST 2 SEQUENCES. Variants in which differences in amino acid sequence relative to the sequence of SEQ ID NO:10 are attributable to genetic polymorphism (allelic variation among individuals producing the protein) are within the scope of the invention.

In certain aspects, this embodiment of the invention may not include the sequence corresponding to GenBank accession no. AF247788.

Fragments of this embodiment of the invention include, but are not limited to, peptides/polypeptides comprising the cytoplasmic domain (e.g., about amino acid 1 to about amino acid 58 of the sequence of SEQ ID NO:10), the transmembrane domain (e.g., about amino acid 59 to about amino acid 81 of the sequence of SEQ ID NO:10), or the extracellular domain (e.g., about amino acid 82 to about amino acid 231 of the sequence of SEQ ID NO:10), of the LCCR polypeptide of SEQ ID NO:10, or variants thereof. The invention also includes fragments of the polypeptide of SEQ ID NO:10, preferably, fragments comprising at least 5 consecutive amino acids, more preferably, at least 10 or at least 20 consecutive amino acids of the sequence of SEQ ID NO:10, or variant thereof. It will be appreciated that fragments of the invention can be employed as immunogens, in generating antibodies.

Variants and fragments of the invention include, but are not limited to, polypeptides that retain a biological activity of the LCCR polypeptide, for example, the ability to bind ligands on tumor and/or virally infected cells and induce cytotoxicity against them. Polypeptides of the invention can be tested for the ability to bind the such ligands in any suitable assay, such as a conventional binding assay.

The invention also includes derivatives/analogs Of the LCCR polypeptide of SEQ ID NO:10 and variants and fragments thereof. The polypeptides of this embodiment of the invention can also be present as a fusion protein, for example, to facilitate detection or isolation. The polypeptides can bear a detectable label and/or can be bound to a solid support.

The invention includes isolated and purified, or homogeneous, polypeptides of this embodiment of the invention, both recombinant and non-recombinant. The polypeptides can be synthesized chemically using art recognized techniques. The polypeptides of this embodiment of the invention can be used as described below or can be used in the production of antibodies (polyclonal or monoclonal) using standard techniques. The invention includes such antibodies, and binding portions thereof, as well as their use, for example, in detecting the presence of a polypeptide of this embodiment of the invention in a sample (in which case, the antibody can bear a detectable label).

The invention further relates to nucleic acid sequences (DNA or RNA) encoding the sequence of SEQ ID NO:10, or variants, and fragments thereof, or the complements of such encoding sequences. One specific nucleic acid sequence encoding the amino acid sequence of SEQ ID NO:10 is set forth in SEQ ID No:9 (see also FIGS. 11 and 12). DNAs of the invention can be single or double stranded. The nucleic acids can bear a detectable label and/or can be bound to a solid support.

As indicated above, this embodiment of the invention includes encoding sequences (DNA and RNA) and sequences complementary thereto. Such complementary sequences include those that hybridize to a nucleic acid sequence encoding the sequence of SEQ ID NO:10, or variant thereof, or fragment thereof, under conditions of moderate or high stringency (as defined above).

This embodiment of the invention also includes nucleic acids comprising sequences that are at least 60%, 70%, 80%, 90%, 95%, 98%, 99%, or at least 99.9% identical to the sequence of SEQ ID NO:9. The percent identity can be determined by visual inspection and mathematical calculation. Alternatively, the percent identity of two nucleic acids can be determined by comparing sequence information using BLAST 2 SEQUENCES.

The present invention also relates to a recombinant molecule comprising a nucleic acid of this embodiment of the invention, as described above, and to a host cell transformed therewith. Using standard methodologies, well known in the art, a recombinant molecule comprising a vector and a nucleic acid encoding a polypeptide of the invention can be constructed. Vectors suitable for use in the present invention include plasmid and viral vectors. Vectors into which a nucleic acid can be cloned include any vectors compatible with transformation into a selected host cell. Such vectors include adenoviral, adeno-associated, retroviral and lentiviral vectors. The nucleic acids of this embodiment of the invention can be present in the vector operably linked to regulatory elements, for example, a promoter.

As indicated above, the recombinant molecule of this embodiment of the invention can be constructed so as to be suitable for transforming a host cell. Suitable host cells include prokaryotic cells and lower and higher eucaryotic cells, such as mammalian cells, such as human cells. The recombinant molecule can be introduced into appropriate host cells by one skilled in the art using a variety of known methods.

The invention further relates to a method of producing a polypeptide of this embodiment. In one aspect, the method comprises culturing the above-described transformed host cells under conditions such that the encoding sequence is expressed and the protein thereby produced.

The identification of LCCR makes possible new therapeutic strategies, for example, immunotherapeutic approaches suitable for use in treating tumors and viral infections, based on the induction of a cytotoxic effect on the immune cells (e.g., NK cells) expressing LCCR. Such strategies can involve the use, for example, of gene therapy or DNA vaccination. Alternatively, soluble forms of the receptor (e.g., the extracellular domain) can be used to abrogate the action of stimulatory ligands in the case of autoimmunne disease treatment.

The invention includes compositions comprising the polypeptides of both embodiments of the invention, nucleic acids, and/or antibodies as described above and a carrier, diluent or excipient, e.g., a pharmaceutically acceptable carrier diluent or excipient. Further, the invention includes kits comprising such polypeptides, nucleic acids and/or antibodies disposed within one or more container means.

Certain aspects of the invention are described in greater detail in the non-limiting Examples that follow (see also Conejo-Garcia et al, Cancer Biology and Therapy 2(4):e112-e117 (2003)). Attention is also directed to U.S. Pat. No. 6,458,350, US Appln. No. 20030147847 and to US Appln. No. 20020187151, the latter describing methods of treating neoplasia that comprise administering ligands for the NKG2D receptor that can be practiced using the ligand (or variant or fragment thereof) disclosed herein.

### EXAMPLE 1

Experimental Details

Identification and characterization of the genomic and cDNA sequences of Letal. The amino acid sequence of the α-1 and α-2 domains of all the known human ligands for the NKG2D receptor (GenBank accession numbers: XM_{—}015542, XM_{—}027342, XM-015533, XM_{—}044229, and XM_{—}029639) were aligned in order to create patterns with the amino acids conserved in at least four of the five sequences and coded by a single exon. Genomic sequences at chromosome 6q25 were translated into the 6 possible open reading frames by using the ORF Finder Program (http://www.ncbi.nlm.nih.gov/gorf/gorf.html) and were scanned for the presence of these patterns with the PattinProt software (http://pbil.ibcp.fr/) (Combet et al, Trends Biochem. Sci. 25(3):147-150 (2000)). Based on these criteria, a sequence was focussed upon that was designated Letal. To search for 5′- and 3′-sequences of the novel gene, the amino acid sequences of ULBP1, ULBP2, and ULBP3 were used to perform a scan algorithm for the detection of genes by using FGENESH+ (http://genomic.sanger.ac.uk/gf/gfs.shtml) (Solovyev et al, Nucleic Acids Res. 27(1):248-250 (1999)) and GeneBuilder (http://125.itba.mi.cnr.it/˜webgene/genebuilder.html) (Milanesi et al, 15(7-8):612-621 (1999)). The primers Letal.F: 5′-CCATACCAGTGAGGGTGAATG-3′ (SEQ ID NO:11) and Letal.R: 5-CCCATGATTCACCTCTCTTGAG-3′ (SEQ ID NO:12) were used to amplify by PCR the complete open reading frame for the predicted gene from the ovarian carcinoma cell line A2008. The putative cleavage sites of the prepropeptide were predicted with SignalP V2.0 (http://www.cbs.dtu.dk/services/SignalP-2.0) (Nielsen et al, Protein Eng. 10(1):1-6 (1997)) and the transmembrane domain with PRED-TMR software (http://o2.db.uoa.gr/PRED-TMR/) (Pasquier et al, Protein Eng. 12(5):381-386 (1999)). Clustalw (http://www.ebi.ac.uk/clustalw) was used to perform alignments and build the phylogenetic tree and PredictProtein (http://maple.bioc.columbia.edu/pp/) (Rost and Sander, J. Mol. Biol. 232(2):584-599 (1993)) was used to predict the secondary structure.

Quantification of Letal by real-time quantitative RT-PCR. Letal expression was analyzed by TaqMan analysis as previously described (Garcia et al, FASEB J 15(10):1819-1821 (2001)). The Letal system consisted of the primers: Letal.F, 5′-CTCAGGATGCTCCTTTGTGACAT-3′ (SEQ ID NO:13); Letal.R, 5′-CTTCACGTTGACAAAACATCTCG-3′ (SEQ ID NO:14), and the probe Letal.P, 5′-(FAM)CCCAGATAAAGACCAGTGATCCTTCCACT (SEQ ID NO:15) (TAMRA)-3′. The cDNA load was normalized to human GAPDH with primers GAPDH F: 5′-CCTGCACCACCAACTGCTTA-3′ (SEQ ID NO:16) and GAPDH R: 5′CATGAGTCCTTCCACGATACCA-3′ (SEQ ID NO:17) and the probe GAPDH.P: 5′(FAM) CCTGGCCAAGGTCATCCATGACAAC (TAMRA)-3′ (SEQ ID NO:18).

Tissues, cell lines and purification of cells. Normal human tissues were obtained from the Cooperative Human Tissue Network (Zhang et al, N. Engl. J. Med. 348(3):203-213 (2003)). A2008 ovarian carcinoma cell line was treated for 24 h with serum-free medium (control) or serum-free medium containing either 10 U/ml interleukin (IL)-1β, 10 ng/ml tumor necrosis factor (TNF-α), 40 ng/ml interferon (IFNγ), 0.5 μg/ml Lypopolyssaccaride (LPS), or 10 ng/ml TNF-α plus 40 ng/ml IFN-γ. 10 ng/ml PMA was kept for 4 hs and retinoic acid for 24, 48, and 72 hs. All the cytokines were from Peprotech (Rocky Hill, N.J.), except retinoic acid (Sigma, St Louis, Mo.). Additionally, cells were cultured in media without glucose for 48 hs or under hypoxia (1.5% O₂) for 16 hs. Fresh peripheral blood lymphocytes were obtained by leukapheresis and elutriation. CD8⁺ cells were prepared by negative selection using the OKT4 antibody (Maus et al, Nat. Biotechnol. 20(2):143-148 (2002)).

Constructs and generation of antibodies. SKOV3 and K562 cells were transduced using retroviral vector MIGR1, generously provided by Warren Pear (University of Pennsylvania). Letal or mock transductants expressing equivalent levels of the green fluorescent protein were sorted and cultured by standard procedures. To demonstrate the binding of Letal to the NKG2D immnoreceptor, Letal⁺/mock-transduced SKOV3 and K562 cells were saturated with 10% mouse serum, and incubated after washing with 3 μg/ml of recombinant human NKG2D/Fc chimera (R & D Systems, Minneapolis, Minn.). Anti-human IgG mAb (G18-145; Pharmingen, San Diego, Calif.) was used to detect the chimerical molecule.

To generate a polyclonal anti-Letal Ab, C57BL/6 mice were immunized at 0, 1 and 2 weeks with 25 μg of Letal cDNA cloned in the pcDNA 3.1 expression vector (Invitrogen). Positive sera were subsequentely confirmed by flow cytometry using different cell lines transfected with the complete Letal Open Reading Frame.

GPI-specific phospholipase-C treatment, ⁵¹Cr release and apoptosis assay. Letal⁺ and Letal⁻ A2780 cells were treated with 2 U/ml GPI-specific phospholipase-C (Sigma) at 37° C. for 1 hr. A standard ⁵¹Cr-release assay was performed using 8,000 ⁵¹Cr-labeled targets/well.

CD8⁺ lymphocyte stimulation and cytokine release. To analyze the effects of Letal on the T-cell proliferation and production of cytokines, a previously described artificial antigen-presenting cell (aAPC) model was used (Maus et al, Nat. Biotechnol. 20(2):143-148 (2002)). Briefly, the system is based on the stable expression of the human low-affinity Fcγ receptor, CD32, on K562 cells (K32 cell line). K32 cell-based aAPCs were additionally transduced with Letal or with the empty vector as described above, irradiated with 100Gy, and washed twice with RPMI medium. Letal⁺-K32 cells or mock transductants were loaded, when indicated, with anti-CD3 (OKT3), or anti-CD3 plus anti-CD28 (mAbs; 9.3) monoclonal antibodies at 0.5 μg/ml for 10 min at room temperature. Loaded &APCs were mixed with CD8⁺ T-cells at a 1:2 ratio and the T-cell concentration was maintained at 0.5×10⁶ cells/ml throughout the culture. Cultures were pulsed with 1 μCi of [³H] thymidine from day 2 to day 5 and incorporated radioactivity was determined using a 1450 Microbeta scintillation counter (Wallac, Turku, Finland). The amounts of secreted IL-2 and IFNγ were determined by commercial ELISA, following the manufacturer's instructions (R & D Systems). Flow cytometry was performed with a FACScalibur (BD Biosciences, San Jose, Calif.). Mouse anti-human monoclonal antibody 149810 (R & D Systems) was used to evaluate the expression of NKG2D.

Results

Analysis of the genomic and cDNA sequences of Letal. The clone NT_{—}007295.3 from the Human Genome Project, containing the sequences of all the ULBPs mapping at chromosome 6q25, was translated into the six possible open reading frames and they were screened for the presence of the pattern “L-Q-X(4)-C-E-X(7)-R-G-S-X(2)-F-X(3)-G-X(2)-F-L-X(6)-W-T”. Based on these criteria, a sequence that was named Lymphocyte Effector cell Toxicity-Activating Ligand (Letal) was focussed upon. Different parts of the cDNA for Letal were bioinformatically deduced and its full-length was amplified from the ovarian carcinoma cell line A2008 (GenBank Accession number: AY069961; submitted on Dec. 12^{th}, 2001). The gene exhibited a 792-bp open reading frame encoding a protein of 263 amino acids in length (FIG. 1A). Letal was found to be identical to a genomic fragment identified as RAET1E, potentially encoding the first 222 amino acids (Radosavljevic et al, Genomics 79(1):114-123 (2002)). Sequence identity of Letal with ULBP1, ULBP2, and ULBP3 ranged from 33.3 to 38.5% (FIG. 1B). As shown in FIG. 1C, molecular phylogenetic analysis confirmed the relatedness of Letal to human ULBPs and to the recently described murine MULT1 (Carayannopoulos et al, J. Immunol. 169(8):4079-4083 (2002)). As with ULBPs, the corresponding protein comprised a class I MHC-like α-1α-2 platform domain. However, it did not encompass glycosylphosphatidyl inositol (GPI) transamidation sites and instead exhibited, uniquely within this group of NKG2D ligands, a cytoplasmic domain.

Letal is a ligand of NKG2D expressed in a variety of normal tissues. To demonstrate that Letal is a ligand for NKG2D, a retroviral system was used to express Letal in erythroleukemia MHC-I^{neg} K562 cells. Expression of the Letal protein was confirmed on transduced cells, but not on mocked transductants, by flow cytometry using serum of Letal-immunized mice (FIG. 1D). To confirm the prediction that Letal does not contain GPI transamidation sites, Letal⁺ K562 cells were treated with GPI-specific phospholipase-C. Instead of decreasing the binding of anti-Letal Ab, enzymatic treatment resulted in stronger staining in three different experiments, suggesting the cleavage of molecules that interfere with the exposure of Letal epitopes, and confirming the predicted transmembrane structure of Letal. To evaluate the binding of NKG2D to transduced Letal, a recombinant soluble NKG2D-Fc fusion protein was used, containing the ectodomain of the immunoreceptor (Phe78 through Val216). Using a monoclonal antibody against human IgG, markedly stronger staining was observed in Letal⁺ K562 and Letal⁺ SKOV3 cells than in mock-transductants by flow cytometry (FIG. 1D), confirming that Letal is a ligand for NKG2D. Treatment with phospholipase C decreased binding of sNKG2D to Letal⁺ K562 cells, although binding was higher than to control cells. No significant decrease was observed with Letal⁺ SKOV3 cells. These results indicate that cleavage of other GPI-anchored NKG2D ligands, but not Letal, accounted for the difference in sNKG2D binding observed between treated and control Letal⁺ K562 cells.

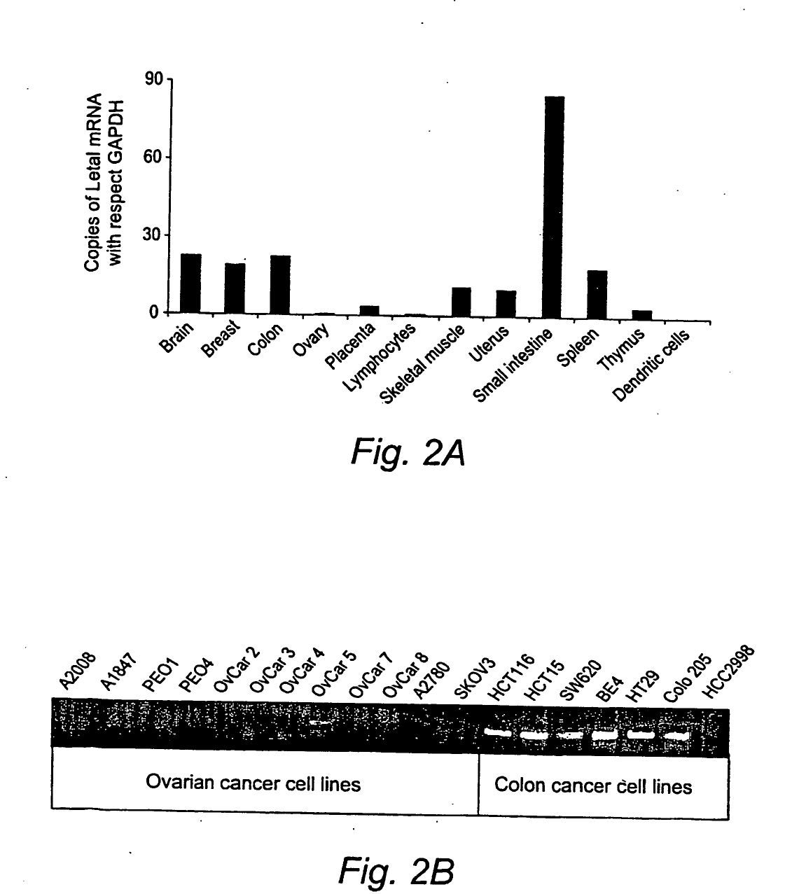

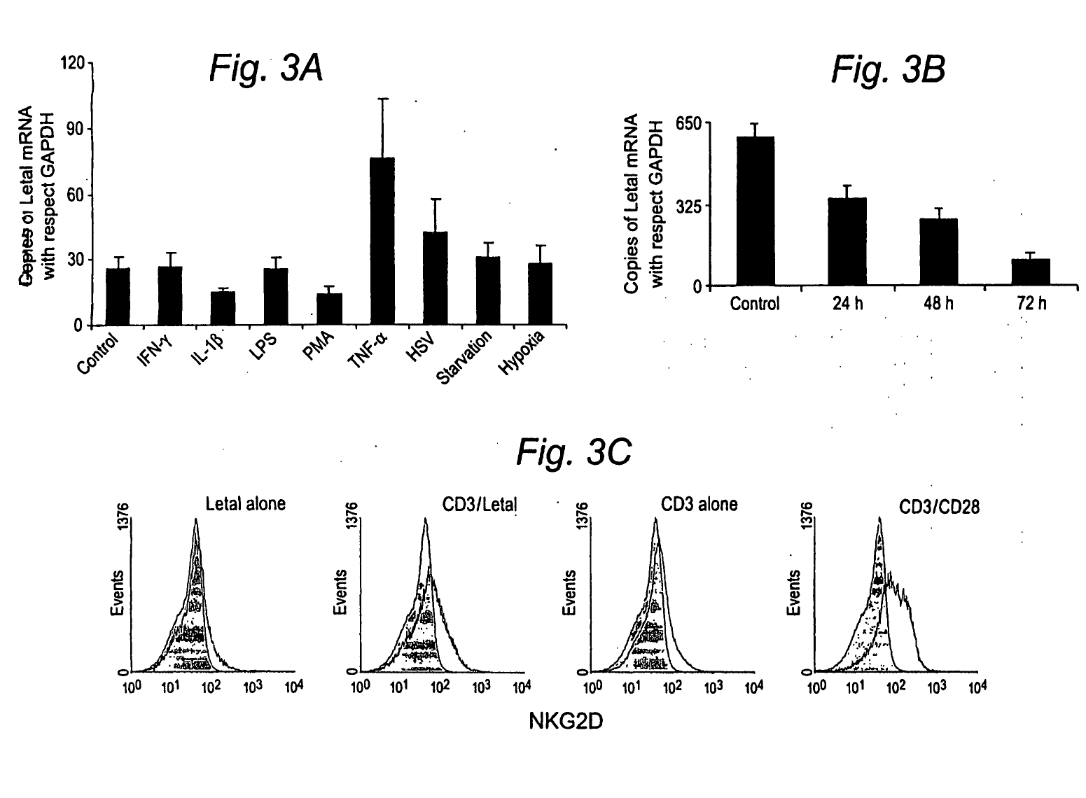

Using real-time quantitative PCR (TaqMan), abundant Letal transcripts were detected in normal small intestine by, and at lower levels in normal brain, breast, colon, spleen, skeletal muscle, uterus, thymus, placenta, blood lymphocytes and ovary (FIG. 2A). RT-PCR analysis indicated the presence of Letal mRNA in most colon cancer cell lines tested, but only in two out of fifteen ovarian carcinoma cell lines (FIG. 2B). A shorter splicing variant encoding for a protein lacking 36 amino acids from the α-1 domain, corresponding to the GenBank Entry: AY054974, was found in 4 cancer cell lines. However, Letal was invariably the predominant form. No expression in immature dendritic cells was detected. Basal Letal mRNA levels increased 1.6-fold and 3-fold in A2008 cells upon infection with Herpes simplex virus or addition of TNF-α, respectively, whereas other inflammatory cytokines, hypoxia or starvation had little to no effect on Letal expression (FIG. 3A). Human MICA/B and mouse RAE-1 family members are upregulated by 48-72 hr treatment with retinoic acid (RA) (Cerwenka et al, Immunity 12(6):721-727 (2000), Jinushi et al, Int. J. Cancer 104(3):354-361 (2003)). Surprisingly, a progressive decrease of Letal mRNA expression was found after treatment of A2008 cells with RA, which was maximum (6-fold) after 72 hr stimulation, suggesting that signals inducing transcriptional activation of NKG2D ligands are markedly different for each molecule (FIG. 3B).

Sustained Letal engagement increases the expression of NKG2D in CD8⁺ lymphocytes. It has been reported that MICA engagement for up to 48 hr causes downregulation of NKG2D and, in turn, impairment of T-cell activation (Groh et al, Nature 419(6908):734-738 (2002)). To determine whether Letal influences NKG2D expression, K32 artificial antigen-presenting cells (aAPCs) (K562 cells transfected with human CD32) were transduced (Maus et al, Nat. Biotech. 20:143 (2002)) with Letal⁺ or control retrovirus, irradiated and cultured with peripheral CD8⁺ cells. CD8⁺ T-cells derived from peripheral blood showed a low mean fluorescence intensity staining of NKG2D after co-culture with mock-transduced K32 erythroleukemia cells for 4 days (FIG. 2C). Instead of decreasing the expression of NKG2D, incubation of lymphocytes with Letal⁺ K32 cells resulted in a slight increase (2-fold), suggesting that either a transcriptional mechanism compensates the initial degradation of NKG2D, or that the effects of Letal are different from that of MICA. This increase was more evident after loading Letal⁺ K32 cells with anti-CD3 mAb (3-fold), whereas CD3/CD28 signaling resulted in the highest up-regulation (4.5-fold increase). Culture of CD8⁺ T-cells with Letal⁻ K32 cells loaded with anti-CD3 mAb produced the same result than CD3/Letal stimulation.

Letal induces T-cell receptor-dependent proliferation and Tc1 polarization in CD8⁺ lymphocytes. To determine whether Letal influences lymphocyte activation, control or Letal⁺ K32 aAPCs were loaded with anti-CD3 and/or anti-CD28 mAb, and incubated for 2 to 5 days with peripheral CD8⁺ cells. Proliferation was similar in the presence of CD3 mAb/Letal and CD3/CD28 mAbs (FIG. 3A). Anti-CD3/Letal stimulation increased day-2 production of IL-2 27-fold and day-3 IFN-γ secretion 83-fold compared to CD3 stimulation alone (FIGS. 3B and 3C). Notably, CD8⁺ cell expansion was markedly lower with CD3 stimulation in the absence of Letal in three independent experiments (29% fewer CD8⁺ cells at day 5). No activation in the absence of anti-CD3 mAb was observed. Taken together, these data demonstrate that Letal is a potent costimulatory molecule for the αβ T-cell receptor, enhancing CD8⁺ cell proliferation and inducing Tc1 responses.

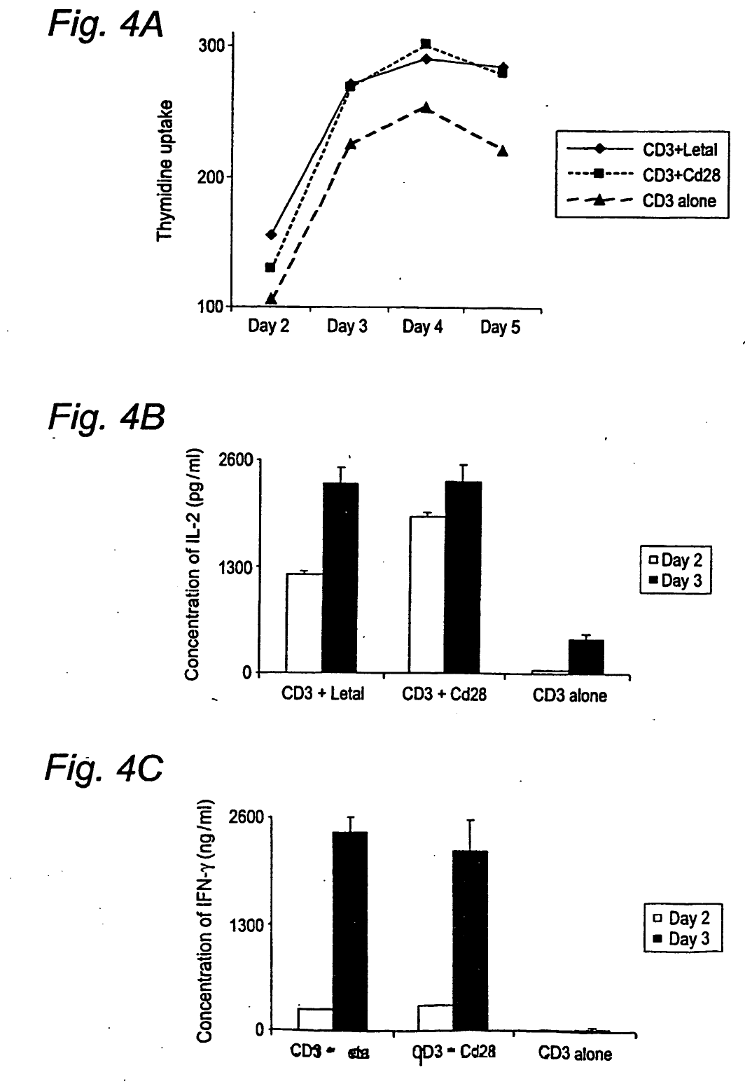

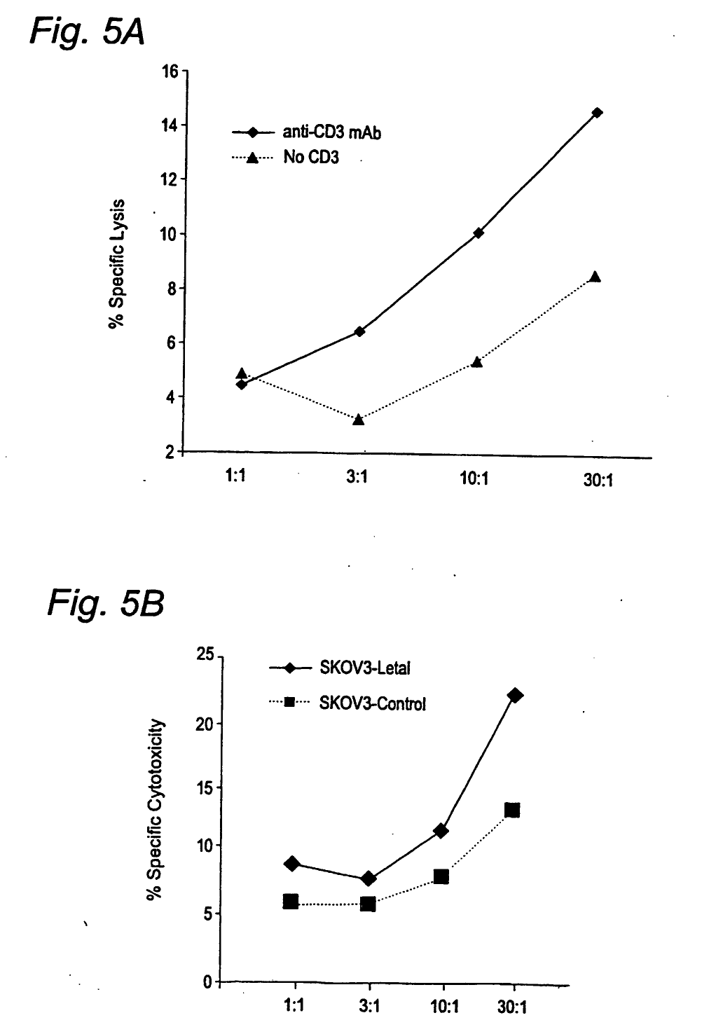

Expression of Letal induces the killing of cancer cells by CD8⁺ and NK cells. Letal-induced cytotoxic effects were next analyzed in a redirected lysis experiment of K562 cells. As expected, CD3/Letal-activated CD8⁺ cells effectively killed MHC-I^{neg} K562 erythroleukemia cells bearing anti-CD3 antibody. In the absence of a TCR signal, Letal engagement alone was markedly less effective (FIG. 4A), while Letal alone-activated lymphocytes failed to kill K562 cells.

For analysis of the NK-dependent anti-tumor immune response, the cytotoxicity of NK cells against Letal⁺ or control SKOV3 chemoresistant ovarian carcinoma cells was compared. Letal expression increased killing of SKOV3 cells by IL-15 activated NK effectors (FIG. 4B), while untreated NK cells could not kill tumor cells efficiently.

Summarizing, the foregoing study resulted in the characterization of the first human transmembrane NKG2D ligand lacking a α-3 domain (Letal). Letal is expressed by tumors and acts as a costimulatory ligand promoting CTL activation, expansion, type-1 polarization and cytotoxicity. Moreover, Letal is directly involved in the activation of NK cell-mediated anti-tumor cytotoxicity. Letal maps to chromosome 6q25, and is identical to a suggested partial sequence lacking 41 amino acids, found through a previous analysis of genomic sequences around the ULBP cluster (Radosavljevic et al, Genomics 79(1):114-123 (2002)). Like the ULBPs, the corresponding Letal protein contains a class I MHC-like α-1α-2 platform domain. However, Letal differs by exhibiting transmembrane and cytoplasmic domains. The highest sequence identity between Letal and a ULBP protein is 38.5%. Interestingly, a splicing variant of Letal has been found in most colon cancer cell lines evaluated. The corresponding peptide lacks 36 amino acids from the α-1 domain and corresponds to an unpublished GenBank entry named as retinoic acid early inducible (RAE)-1-like transcript 4. To the contrary, it has been demonstrated that, instead of being up-regulated by retinoic acid, Letal is down-regulated. This, taken together with the low sequence identity between Letal and RAE-1 family members, suggests that the name RAE may be inexact. Given the functional potency of Letal to stimulate effector immune cells and increase NKG2D expression, retinoic acid-dependent mechanisms could be used to control T-cell proliferation and prevent widespread inflammation through Letal down-regulation. Interestingly, in the human, MICA/B are also up-regulated by retinoic acid (Jinushi et al, Int. J. Cancer 104(3):354-361 (2003)). This apparent contradiction may be explained by a differential expression of activating molecules in different cell types. Alternatively, NKG2D may bind different ligands with different affinities. Moreover, little is known about the expression of these proteins in vivo. It is possible that MICA/B up-regulated by retinoic acid are enzymatically cleaved, thus releasing soluble forms that down-regulate NKG2D expression (Groh et al, Nature 419(6908):734-738 (2002)), finally producing the same diminishing effects.

### EXAMPLE 2

Experimental Details

Quantification of Letal by real-time quantitative RT-PCR. Letal expression was analyzed by TaqMan analysis as previously described (Garcia et al, FASEB J. 15:1819-1821 (2001)). The Letal system consisted of the primers: Letal.F, 5′-CTCAGGATGCTCCTTTGTGACAT-3′ (SEQ ID NO:13); Letal.R, 5′-CTTCACGTTGACAAAACATCTCG-3′ (SEQ ID NO:14), and the probe Letal.P, 5′-(FAM)CCCAGATAAAGACCAGTGATCCTTCCACT (TAMRA)-3′ (SEQ ID NO:15). The cDNA load to human GAPDH was normalized with primers GAPDH F: 5′-CCTGCACCACCAACTGCTTA-3′ (SEQ ID NO:16) and GAPDH R: 5′-CATGAGTCCTTCCACGATACCA-3′ (SEQ ID NO:17) and the probe GAPDH.P: 5′(FAM) CCTGGCCAAGGTCATCCATGACAAC (SEQ ID NO:18) (TAMRA)-3′. The expression of phospholipase-A2 (GLPD1) was quantified using the SYBR Green Master Mix kit (Applied Biosystems) and primers Ph.F: 5′-GCAATGATGTACTGTCTCTTTTGGA-3′ (SEQ ID NO:19) and Ph.R: 5′-CAACCTCAGCCAAGTAACGGTAG-3′ (SEQ ID NO:20).

Tissues, cell lines and purification of cells. Normal human tissues were obtained from the Cooperative Human Tissue Network. Ovarian tumor specimens were obtained from the University of Turin, Italy (Zhang et al, N. Engl. J. Med. 348:203-213 (2003)). For the analysis of tumor-infiltrating lymphocytes, ovarian tumors were minced and digested with collagenase A (Roche, Mannheim, Germany), and cell sorting was performed on a MoFlo cell sorter (Cytomation, Fort Collins, Colo.) with a proportion of the filtered suspension. Fresh peripheral blood lymphocytes were obtained by leukapheresis and elutriation. CD8⁺ cells were prepared by negative selection using the OKT4 antibody (Maus et al, Nat. Biotechnol. 20:143-148 (2002)).

Constructs and generation of antibodies. K562 cells were transduced using retroviral vector MIGR1, generously provided by Warren Pear (University of Pennsylvania). Letal or mock transductants expressing equivalent levels of the green fluorescent protein were sorted and cultured by standard procedures.

To generate a polyclonal anti-Letal Ab, C57BL6 mice were immunized at 0, 1 and 2 weeks with 25 μg of Letal cDNA cloned in the pcDNA 3.1 expression vector (Invitrogen). Positive sera were subsequentely confirmed by flow cytometry using different cell lines transfected with the complete Letal Open Reading Frame.

Apoptosis assay. The percentage of apoptotic cells was determined after 17 h incubation with 50 μM cisplatin or 18 hs exposure to 0.1 μg/ml anti-CD95 mAb (EOS9.1; Pharmingen) by using the TACS annexin-V apoptosis detection kit, according to the manufacturer's instructions (R & D Systems). Fas expression was determined by flow cytometry by using EOS9.1 as a primary Ab and a PE-labeled anti-mouse IgM (R6-60.2; Pharmingen) as a second Ab.

CD8⁺ lymphocyte stimulation, cytokine release and glucose metabolism analysis. To analyze the effects of Letal on the T-cell proliferation and production of cytokines, a previously described artificial antigen-presenting cell (aAPC) model was used (Maus et al, Nat. Biotechnol. 20:143-148 (2002)). Briefly, the system is based on the stable expression of the human low-affinity Fcγ receptor, CD32, on K562 cells (K32 cell line). K32 cell-based aAPCs were additionally transduced with Letal or with the empty vector as described above, irradiated with 100Gy, and washed twice with RPMI medium. Letal⁺-K32 cells or mock transductants were loaded, when indicated, with anti-CD3 (OKT3), or anti-CD3 plus anti-CD28 (mAbs; 9.3) monoclonal antibodies at 0.5 μg/ml for 10 min at room temperature. Loaded aAPCs were mixed with CD8⁺ T-cells at a 1:2 ratio and the T-cell concentration was maintained at 0.5×10⁶ cells/ml throughout the culture. Cultures were pulsed with 1 μCi of [³H] thymidine from day 2 to day 5 and incorporated radioactivity was determined using a 1450 Microbeta scintillation counter (Wallac, Turku, Finland). The amounts of secreted IL-2 and IFN-γ were determined by commercial ELISA, following the manufacturer's instructions (R & D Systems). Flow cytometry was performed with a FACScalibur (BD Biosciences, San Jose, Calif.). Mouse anti-human monoclonal antibody 149810 (R & D Systems) was used to evaluate the expression of NKG2D. Glut-1 intracellular staining and glucose uptake were performed exactly as previously described (Frauwirth et al, Immunity 16:769-777 (2002)).

Laser capture microdissection and immunohistochemistry. Hematoxylin-labeled tumor islands were microdissected from six μm thick cryosections using the μCUT Laser-MicroBeam System (SL Microtest, Jena, Germany), according to the manufacturer's instructions. RNA was immediately extracted from captured tissue by using the PicoPure RNA Isolation kit (Arcturus, Mountain View, Calif.). Immunohistochemistry was performed exactly as previously described (Zhang et al, N. Engl. J. Med. 348:203-213 (2003)).

Results

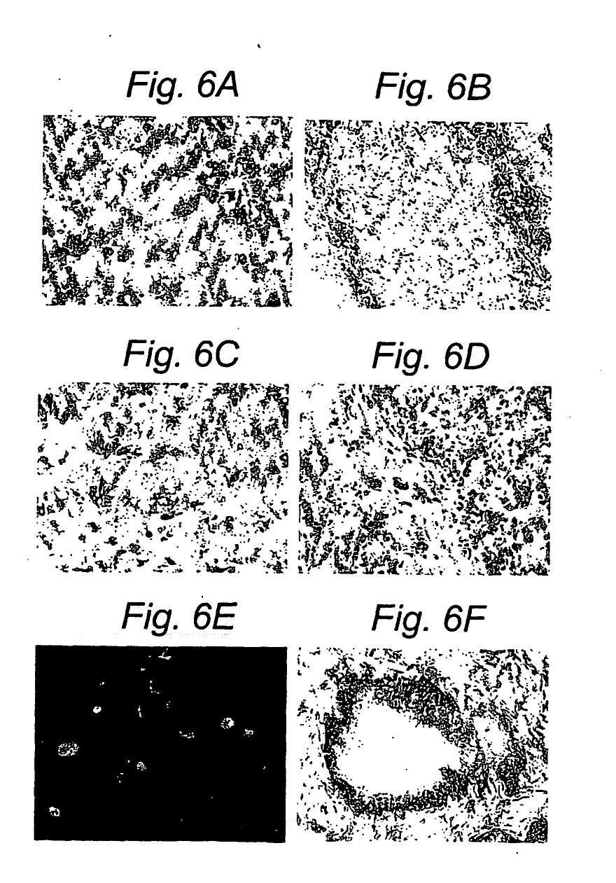

CD8⁺ T-cells represent the predominant NKG2D⁺ population in advanced ovarian carcinomas. The presence of total leukocytes in 100 snap-frozen specimens of ovarian carcinomas was first evaluated by immunohistochemistry. CD45⁺ leukocytes were detected in different proportions within tumor-cell islets, in stroma, or both. CD45⁺ cells represented up to 25% of total cells in selected specimens (FIG. 6A). Next, the expression of NKG2D by tumor-infiltrating leukocytes was examined. More than 50% of tumor islets in these specimens were infiltrated by NKG2D⁺ cells, which, in average, represented 15% of the total leukocytes in stage III tumors (FIG. 6B). The relative contribution of different effector cell types, i.e. CD8⁺ and NK cells, in these tumors was investigated. The vast majority of effector cells were found to be CD8⁺ T-cells (FIG. 6C), while NK cells were scarcely represented in most specimens analyzed (FIG. 6D), suggesting a predominant role of T-cell mediated responses in immunosurveillance against established tumors. Interestingly, lower stage tumors contained significantly fewer infiltrating lymphocytes than advanced ovarian carcinomas, suggesting that a certain degree of invasion and dedifferentiation is necessary to trigger a sustained immune response.

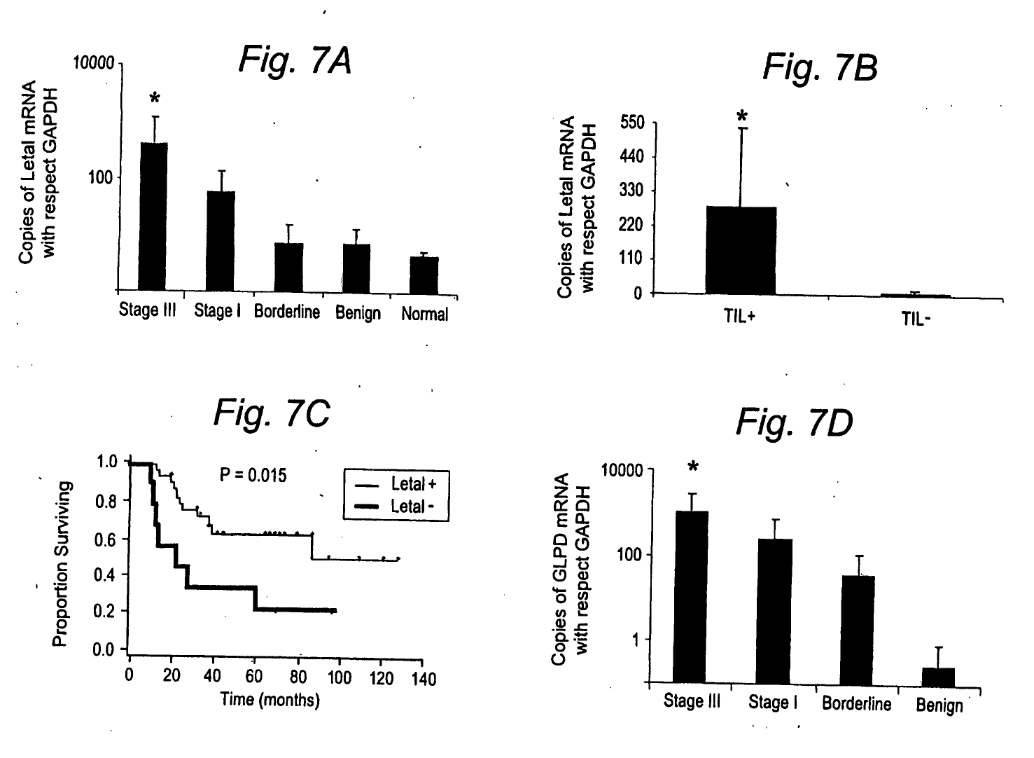

CD8⁺ T-cells infiltration is associated with Letal overexpression in human advanced ovarian carcinomas. Since ovarian cancer progression is associated with an increasing number of infiltrating lymphocytes, Letal expression during tumor progression was next analyzed in 48 ovarian neoplasms and control postmenopausal ovaries. Strong immunostaining for Letal was detected in tumor islets of stage III carcinomas (FIG. 6E, 6F). A positive signal was also observed in select tumor-inflitrating leukocytes within peritumoral stroma. Using Real-Time PCR, it was found that expression of Letal mRNA was low in normal ovaries and benign or low-malignant potential (borderline) tumors, whereas stage I to III ovarian carcinomas exhibited markedly higher expression (higher Letal mRNA expression in malignant tumors (stage III and stage I; n=38) vs borderline and benign tumors, plus normal post-menopausal ovaries (n=10; p=0.02); higher Letal mRNA expression in stage III tumors (n=29) vs stage I, borderline and benign tumors, plus normal post-menopausal ovaries (n=19; p<0.05); FIG. 7A). The average Letal mRNA levels in stage I carcinomas were 10-fold higher compared to non-malignant specimens, whereas Letal expression was 100-fold higher in stage III compared to stage I cancers.

T-cells infiltrate tumor islets (intratumoral T-cells) in approximately 55% of ovarian cancers, while T-cells are exclusively detected in peritumoral stroma in the remainder (zhang et al, N. Engl. J. Med. 348:203-213 (2003)). Simultaneous stimulation of the T-cell receptor and Letal induces proliferation of cytotoxic lymphocytes in vitro (Conejo-Garcia et al, Cancer Biol. Ther. 2 available online). To investigate whether Letal plays any role in the expansion of intratumoral T-cells in vivo, Letal levels in tumor islets showing intratumoral T cells and tumor islets lacking intratumoral T-cells were measured, using laser capture microdissection to procure highly pure samples of tumor islets. TaqMan analysis of 19 different stage III specimens revealed a 30-fold higher Letal mRNA expression in islets infiltrated by T-cells compared to islets lacking T-cells (P=0.041; FIG. 7B). Since most ovarian tumors express MHC-I by immunohistochemistry (Kooi et al, Cell Immunol. 174:116-128 (1996)) and flow cytometry, these data suggest that Letal may be involved in the enrichment of T-cells in tumor islets.

Because, patients with ovarian cancer whose tumors exhibit T-cells infiltrating tumor islets (TILs) experience better outcome (Zhang et al, N. Engl. J. Med. 348:203-213 (2003)), the survival of patients with stage-III ovarian cancer was analyzed based on the expression of Letal mRNA. There were significant differences in the distribution of overall survival (log-rank test; P=0.015; FIG. 7C). Patients whose tumors expressed Letal mRNA had a median overall survival of 37 months (n=29), as compared with 20 months among patients with Letal-negative tumors (n=9). The five-year overall survival rate was 41% among patients whose tumors expressed Letal mRNA but only 22% among patients whose tumors were Letal-negative. Thus, although expression of Letal increases in late stage, it appears to play a protective role in ovarian carcinoma.

It has been reported that engagement of soluble forms of NKG2D ligands causes downregulation of NKG2D and, in turn, impairment of T-cell activation (Groh et al, Nature 419:734-738 (2002)). To test whether different glycosylphosphatidylinositol (GPI)-anchored NKG2D ligands (Cosman et al, Immunity 14:123-133 (2001)) may be secreted by enzymatic cleavage in vivo and impair the expression of NKG2D, the expression of the GPI-specific phospholipase-D (GLPD1) was analyzed in the same tumor specimens. As determined by TaqMan, GLPD1 mRNA levels were significantly higher in malignant (stage I or III) than in benign tumors (P=0.04; FIG. 2D). This data suggests a possible mechanism of immune evasion which can counteract of the immunostimulatory effect of Letal.

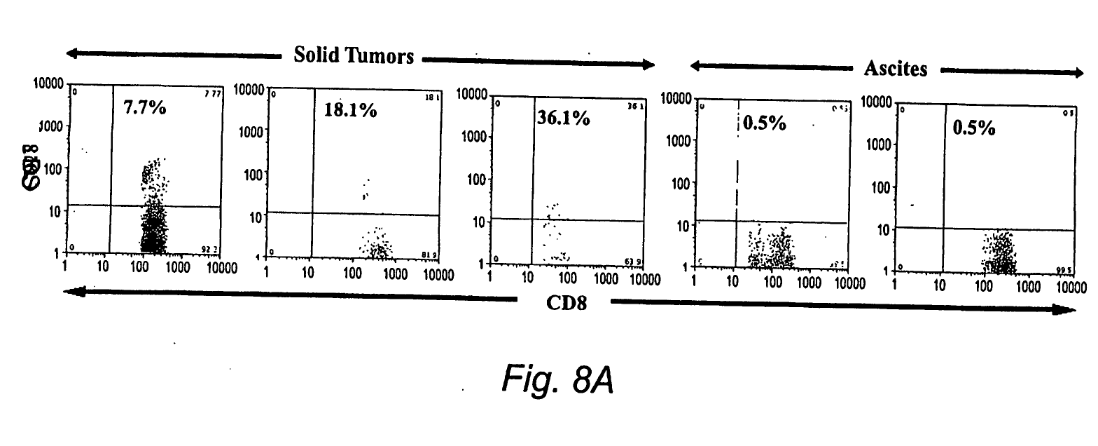

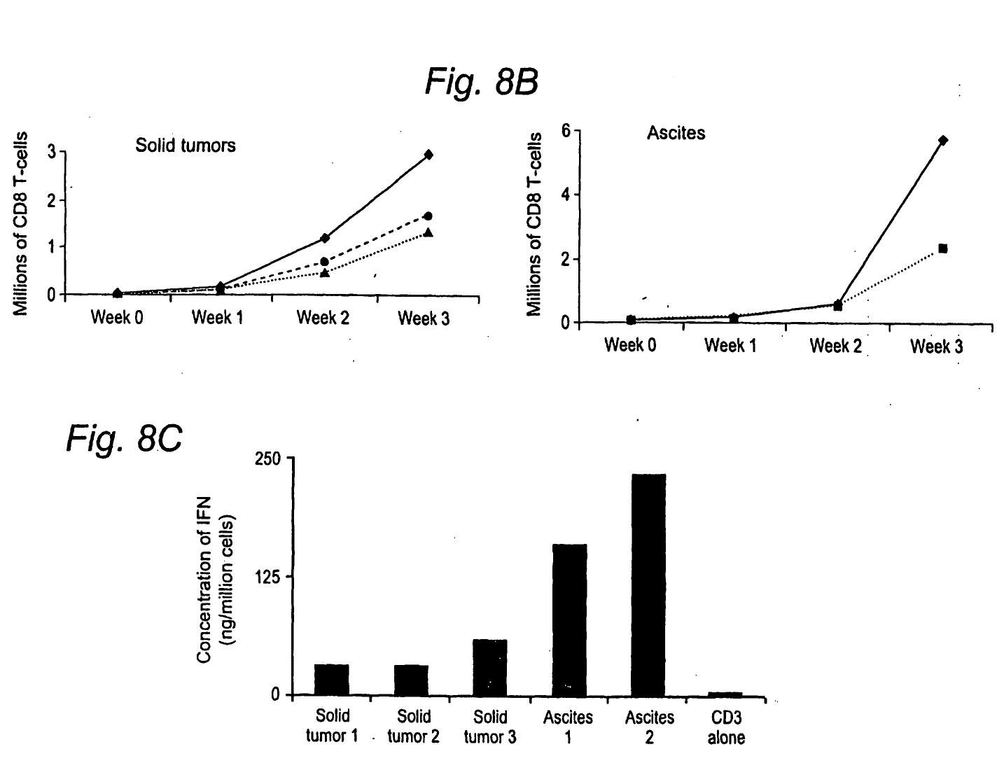

Tumor-infiltrating CD8⁺ lymphocytes do not express CD28 but can be expanded through CD3/Letal engagement. A significant proportion of peripheral effector CD8⁺ cells are known to be negative for the costimulatory molecule CD28, thus antigen-induced proliferative response may be impaired, even after addition of exogenous IL-2, or rely substantially on alternate costimulatory receptors (Azuma et al, J. Immunol. 150:147-1159 (1993)). The expression of CD28 on CD8⁺ lymphocytes from three dissociated ovarian tumors and two tumor ascites specimens was analyzed. Diminished or completely absent expression of CD28 was observed on greater than 80% of tumor-infiltrating cytotoxic lymphocytes, as well as in the vast majority of CD8⁺ lymphocytes from tumor ascites (FIG. 8A). As tumor-infiltrating leukocytes express NKG2D by immunohistochemistry (FIG. 6B). An investigation was made as to whether Letal engagement could compensate for the absence of CD28 in CD8⁺ CD28⁻ lymphocytes sorted from the same specimens. As shown in FIG. 8B, CD3/Letal stimulation delivered through the K562 artificial antigen-presenting system induced a sustained proliferation of CD8⁺ cells from all specimens for at least 3 weeks. Additionally, secretion of IFN-γ was dramatically increased with respect to activation by TCR-CD3 alone (FIG. 8C). Thus, Letal provides an important tumor-associated costimulatory molecule recognized by tumor-associated CD28⁻ CTL. The above findings collectively support an important role of Letal in the intraumoral expansion of TILs in ovarian carcinoma.

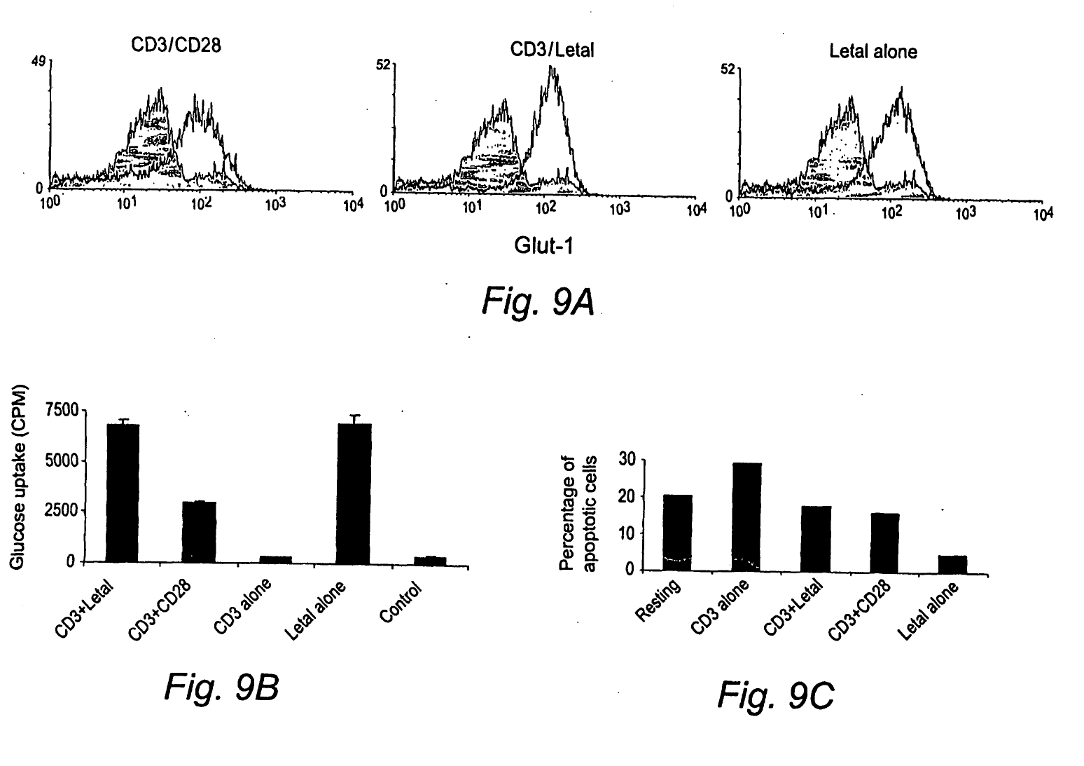

Letal signaling increases glucose transporter expression and glucose uptake during T-cell activation. It has been recently reported that lymphocyte activation through CD28 costimulation increases glycolytic flux (Frauwirth et al, Immunity 16:769-777 (2002)). To test whether Letal may have a similar effect during T-cell activation, peripheral CD8⁺ T-cells were stimulated for 20 hr with anti-CD3, anti-CD3/anti-CD28, anti-CD3/Letal, or Letal alone and the expression of the glucose transporter Glut-1 was analyzed by flow cytometry. Activation by cross-linking the TCR/CD3 complex altered Glut-1 expression only in 11% of the cells (FIG. 9A). In contrast, stimulation with anti-CD3/Letal or Letal alone led to a dramatic induction of Glut-1 expression, which was similar to that induced by CD3/CD28 costimulation.

Glucose uptake rates were next measured with [³H]-2-deoxyglucose in cells stimulated as described above. As expected, anti-CD3/anti-CD28 increased glucose uptake to previously reported levels, while CD3 alone had little to no effect. Interestingly, the uptake rate increase was even more apparent in CD3/Letal and Letal alone-stimulated cells (FIG. 9B). Thus, a signal transduction induced by Letal alone prepares lymphocytes for the increased metabolic demands associated with immune responses.

The studies in ovarian cancer indicated that the presence of intratumoral T cells strongly predicts prolonged remission following cytotoxic chemotherapy (Zhang et al, N. Engl. J. Med. 348:203-213 (2003)). Cytotoxic chemotherapy can, however, deplete tumor-specific effector T-cells (Lee et al, Nat. Med. 5:677-685 (1999)). Because the glycolytic pathway is implicated in lymphocyte survival (Plas et al, Nat. Immunol. 3:515-521 (2002)), a determination was made as to whether Letal could protect T-cells from early apoptotic death induced by genotoxic drugs. Incubation with 50 μM cisplatin for 17 hr produced a higher percentage of apoptotic cells in lymphocytes pre-stimulated for 3 days with anti-CD3 than in resting lymphocytes. In contrast, apoptosis of CD3/Letal and CD3/CD28 pre-stimulated CD8⁺ cells was markedly lower (FIG. 9C). Furthermore, T-cells pre-stimulated with Letal alone showed markedly stronger protection from platinum-induced apoptosis. Collectively, the above data demonstrate that Letal engagement protects CD8⁺ T-cells from apoptosis induced by TCR-dependent mitogenic signals and genotoxic drugs.

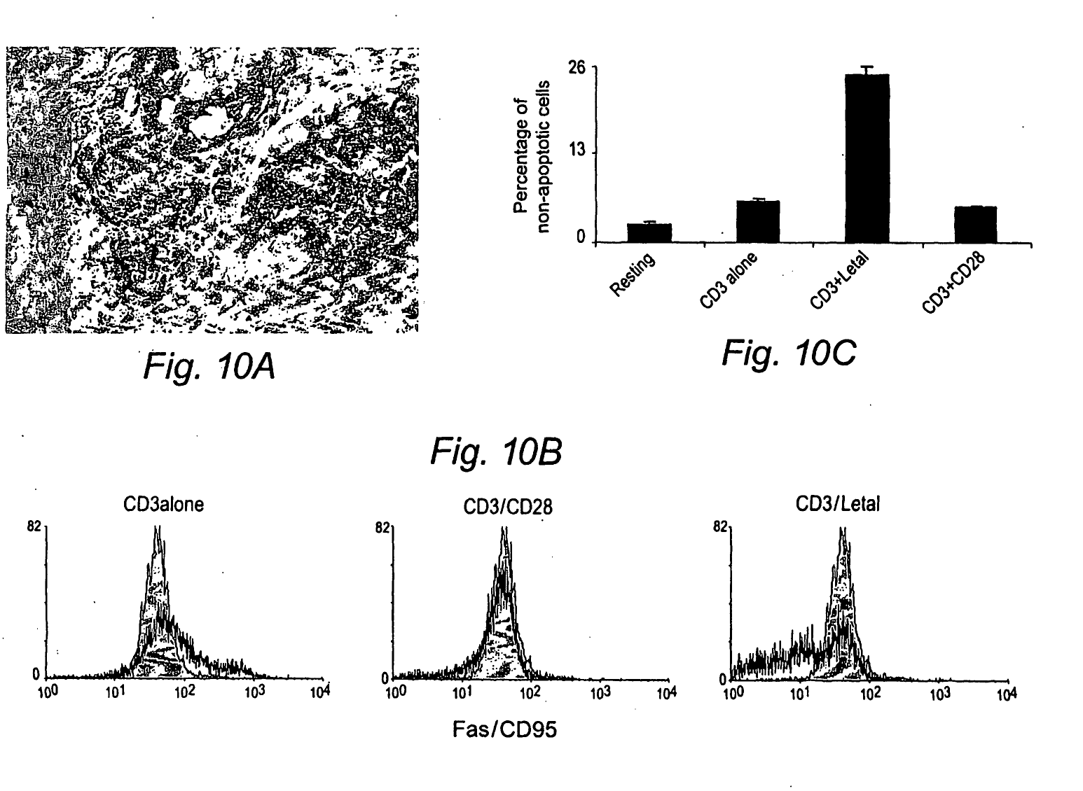

Letal engagement protects CD8⁺ T-cells from FasL-induced apoptosis. Ovarian carcinomas harbor abundant intratumoral T cells and the latter exhibit evidence of activation and are associated with dramatically improved clinical outcome (Zhang et al, N. Engl. J. Med. 348:203-213 (2003)). Because ovarian carcinomas express FasL and other lymphocyte-depleting death ligands (Rabinowich et al, J. Clin. Invest. 101:2579-2588 (1998)), the prevalence of FasL expression and its impact on TILs was determined in 42 stage III ovarian carcinoma specimens. Strong FasL immunoreactivity was detected in tumor cells in areas of selected specimens (FIG. 10A), as well as in a proportion of stromal leukocytes. To test whether Letal promotes the generation of activated T-cell subpopulations that are able to resist the pro-apoptotic tumor microenvironment, peripheral CD8⁺ T-cells were stimulated for 4 days with CD3/Letal, CD3/CD28, or anti-CD3 alone, and expression of Fas was measured by flow cytometry. As shown in FIG. 10B, Fas expression was markedly higher in T-cells stimulated with anti-CD3 mAb compared to CD3/CD28 costimulated T-cells. These data agree with previous reports on TCR activation-induced apoptosis (Zaks et al, J. Immunol. 162:3273-3279 (1999)). Fas expression in unstimulated lymphocytes progressively increased, reaching similar levels to lymphocytes costimulated with CD3/CD28 on day-4. In contrast, Letal engagement dramatically reduced Fas expression in 60% of T-cells compared to controls, suggesting that Letal engagement protects CD8⁺ lymphocytes from FasL-induced apoptosis. CD8⁺ cells stimulated for 3 days with different ligands were therefore exposed to anti-CD95 agonistic antibody EOS9.1 and (early) apoptosis was quantified by flow cytometry analysis of annexin-V staining. As expected, the percentage of non-apoptotic cells among CD3/Letal-stimulated T-cells was dramatically higher than among T-cells stimulated with CD3/CD28, CD3 alone or control T-cells (FIG. 10C). Therefore, Letal confers CD8⁺ lymphocytes the ability to resist suicidal, fratricidal, and tumor-induced apoptosis induced by tumor death ligands.

Summarizing, the data show that human advanced ovarian carcinoma exhibiting improved outcome and stronger lymphocyte infiltration also show higher levels of the NKG2D ligand Letal. Cytotoxic lymphocytes sorted from these tumors were negative for CD28, but Letal exerted marked costimulatory properties on TCR-mediated proliferation of these cells. Moreover, Letal engagement protects CD8⁺ T-cells from apoptosis induced by TCR-dependent mitogenic signals, tumor death ligands and genotoxic drugs. Since NKG2D is an important activating receptor for CD8⁺ lymphocytes and NK cells in peripheral tissues, these results have marked implications for tumor immunosurveillance. NK cells may be responsible for rejection of tumors at early stages of malignant transformation (Diefenbach et al, Nature 413:165-171 (2001), Cerwenka et al, Proc. Natl. Acad. Sci. USA 98:11521-11526 (2001)). However, CD8⁺ cells are markedly more frequent than NK cells in advanced ovarian carcinoma. The presence of tumor infiltrating T-cells correlates with MHC class-I expression of tumor cells in ovarian cancer (Kooi et al, Cell Immunol. 174:116-128 (1996)). This suggests a predominant role for TCR-dependent immune response against established tumors. The presence of T-cells infiltrating tumor islets is associated with dramatically longer survival and prolonged remission in ovarian carcinoma (Zhang et al, N. Engl. J. Med. 348:203-213 (2003)). Interestingly, significantly higher expression of Letal was found in tumor islets exhibiting accumulation of CD3⁺ cells. Collectively, these findings suggest that immune surveillance against advanced ovarian carcinoma is mainly accomplished through expansion of tumor-specific CTL. Given the role of Letal in promoting the survival, proliferation and cytotoxicity of CD8⁺ cells, tumor-associated Letal may play an important role in promoting the expansion of tumor-specific CTLs in the context of MHC-I expression. It is known that upon repeated stimulation with antigen, particularly in the presence of IL-2, T-cells become susceptible to the induction of Fas-mediated apoptosis (Plas et al, Nat. Immunol. 3:515-521 (2002)). It has been proposed that increased resistance of T cells to apoptosis is a necessary condition for the establishment of chronic inflammatory diseases and is required for the orchestration and endurance of sustained immune responses (Levine et al, Semin. Immunool. 13:195-199 (2001), Westermann et al, Ann. Intern. Med. 135:279-295 (2001)). In lymph nodes, activation-induced T cell apoptosis is inhibited by costimulatory signals provided by professional antigen-presenting cells through CD28, CD7 or some members of the TNF receptor family. Engagement of CD28 involves activation of MAP kinases ERK, p38 and JNK; activation of NF-κB; upregulation of Bcl-2, Bcl-x_{L} and c-FLIP; and downregulation of Fas (Kataoka et al, Curr. Biol. 10:640-648 (2000), Khoshnan et al, J. Imunol. 165:1743-1754 (2000), Kirchhoff et al, Eur. J. Immunol. 30:2765-2774 (2000)). In the periphery, NKG2D serves as one of the most potent costimulatory receptors for CD8⁺ effector lymphocytes. Engagement of NKG2D by Letal was found to markedly decrease expression of Fas and significantly reduced TCR activation-induced apoptosis. The survival-promoting effect of Letal was not restricted to the Fas pathway however, as Letal engagement protected lymphocytes also from death induced by the genotoxic drug cisplatin. This finding has important implications for the effects of chemotherapy on antitumor immune response, as activated lymphocytes become susceptible to toxic metabolites and cytotoxic chemotherapy has been shown to deplete tumor-reactive T cells (Zaks et al, J. Immunol. 162:3273-3279 (1999)). In lymphocytes, glycolytic metabolism may play a critical role in controlling cell survival. Withdrawal of exogenous survival factors results in a decline in cellular ATP, which is due, in part, to decreased expression of Glut-1, the major glucose transporter in lymphocytes (Lee et al, Nat. Med. 5:677-685 (1999)). Major cell survival pathways have been reported to alter the metabolic response of lymphocytes to withdrawal of survival factors; sustain ATP production in mitochondria; increase glucose uptake; and/or enhance glycolysis in the absence of extracellular signals (Lee et al, Nat. Med. 5:677-685 (1999), Vander Heiden et al, Mol. Cell 3:159-167 (1999)). CD8⁺ T-cell activation is accompanied by a dramatic increase in glucose uptake through upregulation of Glut-1. It has been recently reported that CD3/CD28 T-cell costimulation increases glycolytic flux, in a manner similar to that of the insulin receptor (Frauwirth et al, Immunity 16:769-777 (2002)). Letal also induced a dramatic increase in glucose uptake and up-regulation of Glut-1. Thus, NKG2D engagement, similarly to CD3/CD28 costimulation, allows T-cells to anticipate the energetic needs of a sustained immune response and appears to afford pro-survival signals through regulation of the glycolytic pathway. Interestingly, Letal signaling alone could trigger glucose up-take, thus the parallels and differences with the CD28 pathway remain to be established. Important questions follow on the mechanisms accounting for failure of immunosurveillance. It is possible that surveillance eventually selects for immunoresistant tumor variants that are capable of escaping CTL-mediated killing, inducing T-cell apoptosis or unresponsiveness (anergy) (Boon and van Baren, N. Engl. J. Med. 348:252-254 (2003)), or simply dividing faster than CTL can kill. Letal could be then used as a costimulatory ligand to expand ex vivo in a CD28-independent manner apoptosis-resistant tumor reactive T-cells for adoptive transfer using artificial APCs. Given that peripheral effector CD8⁺ cells are mainly CD28^{low/neg}, such approach might offer significant advantage over CD28-based costimulation. The fact that anti-tumor response varies depending on the level of NKG2D ligands that are expressed (Diefenbach et al, Nature 413:165-171 (2001)) supports the notion that expansion of specific T-cells at tumor sites, or protection of them against chemotherapy, can be boosted by engineering cells with higher levels of Letal or using soluble forms of the ligand.

All documents cited above are hereby incorporated in their entirety by reference.

What is claimed is:

1. A polypeptide comprising the sequence of SEQ ID NO:8, or variant or fragment thereof.

2. The polypeptide according to claim 1 wherein said polypeptide comprises the sequence of SEQ ID NO:8 or variant thereof that shares at least 50% identity with the sequence of SEQ ID NO:8.

3. The polypeptide according to claim 2 wherein said variant shares at least 70% identity with the sequence of SEQ ID NO:8.

4. The polypeptide according to claim 3 wherein said variant shares at least 90% identity with the sequence of SEQ ID NO:8.

5. The polypeptide according to claim 1 wherein said polypeptide comprises the sequence of SEQ ID NO:8 or fragment thereof of at least 5 contiguous amino acids.

6. The polypeptide according to claim 5 wherein said polypeptide comprises the sequence of SEQ ID NO:8 or fragment thereof of at least 20 contiguous amino acids.

7. A polypeptide comprising at least one of the signal peptide, the α-1 domain, the α-2 domain, the transmembrane domain and the cytoplasmic domain of the sequence of SEQ ID NO:8 or variant thereof.

8. The polypeptide according to claim 7 wherein said polypeptide comprises about amino acid 29 to about amino acid 225 of the sequence of SEQ ID NO:8.

9. An isolated nucleic acid that encodes the polypeptide according to claim 1, or a nucleic acid complementary thereto.

10. The nucleic acid according to claim 9 wherein said nucleic acid comprises the sequence of nucleotides shown in SEQ ID NOs:1-4 that encode the sequence of SEQ ID NO:8, or a nucleic acid complementary thereto.

11. The nucleic acid according to claim 9 wherein said nucleic acid comprises a nucleotide sequence sharing at least 50% identity with the nucleotide sequence set forth in SEQ ID NOs:1-4 that encodes the amino acid sequence set forth in SEQ ID NO:8.

12. A construct comprising a vector and the nucleic acid according to claim 9.

13. The construct according to claim 12 wherein said vector is a viral vector.

14. The construct according to claim 12 wherein said nucleic acid is operably linked to a promoter.

15. The construct according to claim 14 wherein said promoter is a tumor specific promoter.

16. A host cell comprising the construct according to claim 12.

17. The host cell according to claim 16 wherein said host cell is a mammalian cell.

18. A method of producing a polypeptide comprising culturing the host cell according to claim 16 under conditions such that said nucleic acid is expressed and said polypeptide is thereby produced.

19. A therapeutic method comprising administering to a patient in need thereof the polypeptide according to claim 1 in an amount sufficient to stimulate effector immune cells of said patient.

20. The method according to claim 19 wherein said patient bears a tumor.

21. The method according to claim 20 wherein a nucleic acid encoding said polypeptide according to claim 1 is introduced into tumor cells of said patient.

22. The method according to claim 19 wherein said patient has a viral infection.

23. An antibody specific for the polypeptide of claim 1, or binding fragment thereof.

24. A polypeptide comprising the sequence of SEQ ID NO:10, or variant or fragment thereof.

25. A polypeptide comprising at least one of the transmembrane, cytoplasmic and extracellular domains of the sequence of SEQ ID NO:10 or variant thereof.

26. An isolated nucleic acid that encodes the polypeptide according to claim 24, or a nucleic acid complementary thereto.

27. A construct comprising a vector in the nucleic acid according to claim 26.

28. A host cell comprising the construct according to claim 27.

29. A method of producing a polypeptide comprising culturing the host cell according to claim 28 under conditions such that said nucleic acid is expressed and said polypeptide is thereby produced.

30. An antibody specific for the polypeptide of claim 24.


SEQUENCE LISTING (21 sequences)

SEQ ID NO: 1 - DNA, 148 bp, Artificial Sequence
  Description of Artificial Sequencehuman ligand for NKG2D receptor
accataccagtgagggtgaatgtgtacacgcccagcttcctgcctgttactctccacagtatgcgaagaatatccctgacttctagccctgtgcgccttcttttgtttctgctgttgctactaatagccttggagatcatggttggtg

SEQ ID NO: 2 - DNA, 257 bp, Artificial Sequence
  Description of Artificial Sequencehuman ligand for NKG2D receptor
gtcactctctttgcttcaacttcactataaaatcattgtccagacctggacagccctggtgtgaagcgcaggtcttcttgaataaaaatcttttccttcagtacaacagtgacaacaacatggtcaaacctctgggcctcctggggaagaaggtatatgccaccagcacttggggagaattgacccaaacgctgggagaagtggggcgagacctcaggatgctcctttgtgacatcaaaccccagataaagaccagt

SEQ ID NO: 3 - DNA, 276 bp, Artificial Sequence
  Description of Artificial Sequencehuman ligand for NKG2D receptor
gatccttccactctgcaagtcgagatgttttgtcaacgtgaagcagaacggtgcactggtgcatcctggcagttcgccaccaatggagagaaatccctcctctttgacgcaatgaacatgacctggacagtaattaatcatgaagccagtaagatcaaggagacatggaagaaagacagagggctggaaaagtatttcaggaagctctcaaagggagactgcgatcactggctcagggaattcttagggcactgggaggcaatgccagaaccgaca

SEQ ID NO: 4 - DNA, 204 bp, Artificial Sequence
  Description of Artificial Sequencehuman ligand for NKG2D receptor
gtgtcaccagtaaatgcttcagatatccactggtcttcttctagtctaccagatagatggatcatcctgggggcattcatcctgttagttttaatgggaattgttctcatctgtgtctggtggcaaaatggtgagtggcaggctggtctctggcccttgaggacgtcttagtctggtaaggactcaagagaggtgaatcatggg

SEQ ID NO: 5 - protein, 244 aa, Artificial Sequence
  Description of Artificial SequenceULBP1
MAAAASPAFLLCLPLLHLLSGWSRAGWVDTHCLCYDFIITPKSRPEPQWCEVQGLVDERPFLHYDCVNHKAKAFASLGKKVNVTKTWEEQTETLRDVVDFLKGQLLDIQVENLIPIEPLTLQARMSCEHEAHGHGRGSWQFLFNGQKFLLFDSNNRKWTALHPGAKKMTEKWEKNRDVTMFFQKISLGDCKMWLEEFLMYWEQMLDPTKPPSLAPGTTQPKAMATTLSPWSLLIIFLCFILAGR

SEQ ID NO: 6 - protein, 246 aa, Artificial Sequence
  Description of Artificial SequenceULBP2
MAAAAATKILLCLPLLLLLSGWSRAGRADPHSLCYDITVIPKFRPGPRWCAVQGQVDEKTFLHYDCGNKTVTPVSPLGKKLNVTTAWKAQNPVLREVVDILTEQLRDIQLENYTPKEPLTLQARMSCEQKAEGHSSGSWQFSFDGQIFLLFDSEKRMWTTVHPGARKMKEKWENDKVVAMSFHYFSMGDCIGWLEDFLMGMDSTLEPSAGAPLAMSSGTTQLRATATTLILCCLLIILPCFILPGI

SEQ ID NO: 7 - protein, 244 aa, Artificial Sequence
  Description of Artificial SequenceULBP3
MAAAASPAILPRLAILPYLLFDWSGTGRADAHSLWYNFTIIHLPRHGQQWCEVQSQVDQKNFLSYDCGSDKVLSMGHLEEQLYATDAWGKQLEMLREVGQRLRLELADTELEDFTPSGPLTLQVRMSCECEADGYIRGSWQFSFDGRKFLLFDSNNRKWTVVHAGARRMKEKWEKDSGLTTFFKMVSMRDCKSWLRDFLMHRKKRLEPTAPPTMAPGLAQPKAIATTLSPWSFLIILCFILPGI

SEQ ID NO: 8 - protein, 263 aa, Artificial Sequence
  Description of Artificial Sequencehuman ligand for NKG2D receptor
MRRISLTSSPVRLLLFLLLLLIALEIMVGGHSLCFNFTIKSLSRPGQPWCEAQVFLNKNLFLQYNSDNNMVKPLGLLGKKVYATSTWGELTQTLGEVGRDLRMLLCDIKPQIKTSDPSTLQVEMFCQREAERCTGASWQFATNGEKSLLFDAMNMTWTVINHEASKIKETWKKDRGLEKYFRKLSKGDCDHWLREFLGHWEAMPEPTVSPVNASDIHWSSSSLPDRWIILGAFILLVLMGIVLICVWWQNGEWQAGLWPLRTS

SEQ ID NO: 9 - DNA, 710 bp, Artificial Sequence
  Description of Artificial SequencecDNA sequence of immune cell receptor LCCR
tttcgagcacatgtgtttttatgagaattatgctgagatagatttctttacatattcatcaatgtctgaagaagttacttatgcagatcttcaattccagaactccagtgagatggaaaaaatcccagaaattggcaaatttggggaaaaagcacctccagctccctctcatgtatggcgtccagcagccttgtttctgactcttctgtgccttctgttgctcattggattgggagtcttggcaagcatgtttcacgtaactttgaagatagaaatgaaaaaaatgaacaaactacaaaacatcagtgaagagctccagagaaatatttctctacaactgatgagtaacatgaatatctccaacaagatcaggaacctctccaccacactgcaaacaatagccaccaaattatgtcgtgagctatatagcaaagaacaagagcacaaatgtaagccttgtccaaggagatggatttggcataaggacagctgttatttcctaagtgatgatgtccaaacatggcaggagagtaaaatggcctgtgctgctcagaatgccagcctgttgaagataaacaacaaaaatgcattggaatttataaaatcccagagtagatcatatgactattggctgggattatctcctgaagaagattccactcgtggtatgagagtggataatataatcaactcctctgcctggtaagtgt

SEQ ID NO: 10 - protein, 231 aa, Artificial Sequence
  Description of Artificial SequenceImmune LCCR cell receptor
MCFYENYAEIDFFTYSSMSEEVTYADLQFQNSSEMEKIPEIGKFGEKAPPAPSHVWRPAALFLTLLCLLLLIGLGVLASMFHVTLKIEMKKMNKLQNISEELQRNISLQLMSNMNISNKIRNLSTTLQTIATKLCRELYSKEQEHKCKPCPRRWIWHKDSCYFLSDDVQTWQESKMACAAQNASLLKINNKNALEFIKSQSRSYDYWLGLSPEEDSTRGMRVDNIINSSAW

SEQ ID NO: 11 - DNA, 21 bp, Artificial Sequence
  Description of Artificial SequencePrimer
ccataccagtgagggtgaatg

SEQ ID NO: 12 - DNA, 22 bp, Artificial Sequence
  Description of Artificial SequencePrimer
cccatgattcacctctcttgag

SEQ ID NO: 13 - DNA, 23 bp, Artificial Sequence
  Description of Artificial SequencePrimer
ctcaggatgctcctttgtgacat

SEQ ID NO: 14 - DNA, 23 bp, Artificial Sequence
  Description of Artificial SequencePrimer
cttcacgttgacaaaacatctcg

SEQ ID NO: 15 - DNA, 29 bp, Artificial Sequence
  Description of Artificial SequencePrimer
cccagataaagaccagtgatccttccact

SEQ ID NO: 16 - DNA, 20 bp, Artificial Sequence
  Description of Artificial SequencePrimer
cctgcaccaccaactgctta

SEQ ID NO: 17 - DNA, 22 bp, Artificial Sequence
  Description of Artificial SequencePrimer
catgagtccttccacgatacca

SEQ ID NO: 18 - DNA, 25 bp, Artificial Sequence
  Description of Artificial SequencePrimer
cctggccaaggtcatccatgacaac

SEQ ID NO: 19 - DNA, 25 bp, Artificial Sequence
  Description of Artificial SequencePrimer
gcaatgatgtactgtctcttttgga

SEQ ID NO: 20 - DNA, 23 bp, Artificial Sequence
  Description of Artificial SequencePrimer
caacctcagccaagtaacggtag

SEQ ID NO: 21 - DNA, 885 bp, Artificial Sequence
  Description of Artificial Sequencehuman ligand for NKG2D receptor
accataccagtgagggtgaatgtgtacacgcccagcttcctgcctgttactctccacagtatgcgaagaatatccctgacttctagccctgtgcgccttcttttgtttctgctgttgctactaatagccttggagatcatggttggtggtcactctctttgcttcaacttcactataaaatcattgtccagacctggacagccctggtgtgaagcgcaggtcttcttgaataaaaatcttttccttcagtacaacagtgacaacaacatggtcaaacctctgggcctcctggggaagaaggtatatgccaccagcacttggggagaattgacccaaacgctgggagaagtggggcgagacctcaggatgctcctttgtgacatcaaaccccagataaagaccagtgatccttccactctgcaagtcgagatgttttgtcaacgtgaagcagaacggtgcactggtgcatcctggcagttcgccaccaatggagagaaatccctcctctttgacgcaatgaacatgacctggacagtaattaatcatgaagccagtaagatcaaggagacatggaagaaagacagagggctggaaaagtatttcaggaagctctcaaagggagactgcgatcactggctcagggaattcttagggcactgggaggcaatgccagaaccgacagtgtcaccagtaaatgcttcagatatccactggtcttcttctagtctaccagatagatggatcatcctgggggcattcatcctgttagttttaatgggaattgttctcatctgtgtctggtggcaaaatggtgagtggcaggctggtctctggcccttgaggacgtcttagtctggtaaggactcaagagaggtgaatcatggg